(19) United States
(12) Patent Application Publication
(10) Pub. No.: US 2022/0354896 A1
(43) Pub. Date: Nov. 10, 2022

(54) COMPOSITIONS AND METHODS FOR THE TREATMENT OF RETINAL DEGENERATION

(71) Applicant: LINEAGE CELL THERAPEUTICS, INC., Carlsbad, CA (US)

(72) Inventors: Igor NASONKIN, Carlsbad, CA (US); Ratnesh SINGH, Carlsbad, CA (US); Francois BINETTE, Carlsbad, CA (US)

(21) Appl. No.: 17/606,990

(22) PCT Filed: Apr. 28, 2020
(86) PCT No.: PCT/US20/30252

§ 371 Date: Oct. 27, 2021

Related U.S. Application Data
(60) Provisional application No. 62/839,748, filed on Apr. 28, 2019.

(51) Int. Cl. A61K 35/30; C12N 5/079; A61P 27/02; A61K 9/00

(52) U.S. Cl. CPC A61K 35/30; C12N 5/0621; A61P 27/02; A61K 9/0019

(57) ABSTRACT

Presented herein are compositions and methods for generating stem cell derived retinal tissue and isolated retinal progenitor cells for use in the treatment of retinal degenerative diseases and disorders.

### CROSS-REFERENCE TO RELATED APPLICATIONS

This application claims priority to International Patent Application No. PCT/US20/30252, filed on Apr. 28, 2020, and to U.S. Provisional Application Ser. No. 62/839,748, filed Apr. 28, 2019, which are hereby incorporated by reference in their entireties.

### STATEMENT REGARDING FEDERALLY SPONSORED RESEARCH OR DEVELOPMENT

This invention was made with government support under SBIR 1R44EY027654 awarded by National Institutes of Health. The government has certain rights in the invention.

### BACKGROUND

Retinal degenerative diseases, which include for example conditions such as age-related macular degeneration (AMD) and retinitis pigmentosa (RP), are a major cause of blindness worldwide. With current advances in genetic testing and ocular imaging, retinal degenerative disease can be identified at early stages. However, at present, there are no adequate treatments available to restore vision following photoreceptor (PR) death. Thus, there is an unmet need for new effective treatments to preserve and restore vision in patients with retinal degeneration (RD).

The present disclosure addresses these and other shortcomings in the field of regenerative therapeutics, vision restoration and vision preservation.

### SUMMARY

Stem cell derived retinal tissue compositions have been developed that are useful for the treatment of retinal diseases or disorders, including preventing the progression of retinal degeneration and vision loss. These stem cell derived retinal tissue compositions may promote the support and survival, regeneration or growth of living cells.

Large quantities of retinal progenitors isolated (dissociated) from hESC-derived retinal tissue useful for manufacturing therapeutics can be generated using the methods described herein and offer a scalable alternative to treatments that involve human fetal retinal tissue.

In some aspects, a pharmaceutical composition for treating or slowing the progression of a retinal degenerative disease or disorder comprises retinal progenitor cells isolated from stem cell derived retinal tissue; and a pharmaceutically acceptable carrier. In other aspects, the cell composition comprises between about 0.5 million and 1.5 million cells. In yet other aspects, the retinal progenitor cells express one or more of the genes OPN, IL6, VEGFA, CXCL12, PTN, Lefty2, FGF9, ctgf, JAG1, NOG, KDR, Nodal, NRG1, hbergf, bmp2, ngfr, gdf11, tgfb1, MDK, cxcr4, sod1, B2M, SDF, CRABP1, SIRT2, SERPINF1, CLU, and BSG.

In some aspects, a method of generating retinal progenitor cells comprises differentiating stem cells into retinal tissue in a medium comprising lectin; and dissociating the retinal tissue to isolate retinal progenitor cells.

In yet other aspects, a method of treating or slowing the progression of a retinal disease or disorder comprises administering a therapeutically effective amount of a pharmaceutical composition comprising retinal progenitor cells isolated from stem cell derived retinal tissue. In some aspects, the retinal progenitor cells express one or more of the genes OPN, IL6, VEGFA, CXCL12, PTN, Lefty2, FGF9, ctgf, JAG1, NOG, KDR, Nodal, NRG1, hbergf, bmp2, ngfr, gdf11, tgfb1, MDK, cxcr4, sod1, B2M, SDF, CRABP1, SIRT2, SERPINF1, CLU, and BSG.

### BRIEF DESCRIPTION OF THE DRAWINGS

For a fuller understanding of the nature and advantages of the present invention, reference should be had to the following detailed description taken in connection with the accompanying drawings, in which:

FIG. 1 shows an image of a developing stem cell derived retinal tissue aggregate (organoid) at month 2-3.

FIG. 2 shows an image of developing stem cell derived retinal tissue aggregates (organoids) at month 2-3 at a size of about 1.6 mm by about 1.62 mm.

FIG. 3 shows an image of developing stem cell derived retinal tissue aggregates (organoids) at month 2-3.

FIG. 4 shows images of an immunohistochemical stained cryosections of stem cell derived retinal tissue (at age 2-3 months) positive for neural retinal progenitor markers CHX10 (VSX2) and PAX6, markers of developing neural retina. Tissue has also been counterstained with with pan-nuclear stain 4′,6-diamidino-2-phenylindole (DAPI).

FIG. 5 shows an immunocytochemical image of the rim of the stem cell derived retinal tissue. Cells are shown staining positive for Recoverin (marker of rod and cone photoreceptors) and THRB2 (Thyroid hormone receptor beta, cone viability and early cone marker), illustrating the developing cone (and rod) photoreceptors. Some of these early photoreceptor cells have double staining for Recoverin and THRB2 (developing cone photoreceptors).

FIG. 6 shows a magnified image of human pluripotent stem cell differentiated tissue at age 4-5 months, with the dark pigmented cells in the center and the visible outer rim protrusions.

FIG. 7 shows the beginning of developing inner and outer segments with the cilia in photoreceptors within the rim about 4-5 months after differentiation using lectin.

FIG. 8 shows the results of karyotypes retinal progenitors isolated from stem cell derived retinal tissue.

FIG. 9 shows images of stem cell derived retinal tissue aggregates and retinal progenitor cells isolated using papain at passage 2 after dissociation from tissue aggregates.

FIG. 10 shows an image of retinal progenitor cells at passage 2 being injected into the epiretinal space of the eye of a rabbit.

FIG. 11 shows images and graphs of the untrasonography results after scanning ocular grafts of retinal progenitors with A- and B-ultrasound waves and a table A, B-wave electroretinogram (ERG: flash flicker) results showing the location of implanted retinal progenitor cells isolated from stem cell derived retinal tissue. Panel H shows the A-wave readings from behind the lens. No negative impacts on the electrophysiological function of rabbit retina, 1 week after ocular (epiretinal) grafting were found.

FIG. 12 shows a diagram and histological image of ex vivo delivery of 3×10⁶ organoid-derived human retinal progenitors into a rabbit eye grafted soon after termination and removal of the eye.

FIG. 13 shows an immunohistochemical image of immunostatined sections of a rabbit eye with human retinal progenitor cells after delivery of the cells into a rabbit eye. The red (HNu) staining can be seen in the graft but not in the rabbit retina showing the human origin of the graft.

FIG. 14 shows a graph of the gradual decline in cone photoreceptor ERG between about day 50 and 150 in a large cohort of PDE6A^{-/-} dogs.

FIG. 15 shows a RetCam image of a graft located close to the peripheral retina.

FIG. 16 shows human embryonic stem cell derived retinal tissue retinal progenitor cells at passage 2, dissociated with papain.

FIG. 17 shows an image of stem cell derived retinal tissue derived from the H1 (WA01) hESC line at between about 2-3 months using methods described herein.

FIG. 18A through FIG. 18C are immunohistochemical images showing the distribution of cell division marker, Ki67 and PAX6 in the neural retina of the rim of stem cell derived retinal tissue at about 2.5 months after initiation of induced differentiation. As shown, Ki67 distribution resembles that in the developing mammalian neural retina (˜9-12 week of human development).

FIG. 19A through FIG. 19C are immunohistochemical images showing the distribution of cell division marker, Ki67 and PAX6 in the neural retina of stem cell derived retinal tissue at about 2.5 months after initiation of induced differentiation. As shown, Ki67 distribution in the manmade artificial retinal tissue developed herein resembles that in the developing mammalian neural retina (˜9-12 week of human development).

FIG. 20A through FIG. 20F are images of immunohistochemically stained hESC-3D retinal tissue (retinal organoid, frozen section) with antibodies to RX (RAX, an eyefield marker), and CRX (cone-rod homeobox, photoreceptor marker), counterstained with pan-nuclear stain 4′,6-diamidino-2-phenylindole (DAPI).

FIG. 21A through FIG. 21F are images of immunohistochemically stained hESC-3D retinal tissue (retinal organoid, frozen section) with antibodies to OTX2 (cone-rod photoreceptors and RPE) and BLIMP1, a photoreceptor progenitor marker), counterstained with pan-nuclear stain 4′,6-diamidino-2-phenylindole (DAPI). Stem cells were induced to differentiate for between 2-2.5 months and show co-localization of BLIMP1[+] and OTX2[+] photoreceptor progenitors in the rim. Photoreceptors are born in the apical side next to RPE (asterisk). A number of OTX2[+] photoreceptor progenitors remain in the central core area and fail to exit.

FIG. 22A through FIG. 22C are images of immunohistochemically stained hESC-3D retinal tissue (retinal organoid, frozen section), at between 2 and 2.5 months after inducing differentiation, with antibodies to NEUROD1 (photoreceptor progenitor and amacrine cell progenitor marker), counterstained with pan-nuclear stain 4′,6-diamidino-2-phenylindole (DAPI).

FIG. 23A through 23C are images of immunohistochemically stained hESC-3D retinal tissue (retinal organoid, frozen section), at between 2 and 2.5 months after inducing differentiation, with antibodies to Calretinin (Calbindin-2, amacrine cell marker), counterstained with pan-nuclear stain 4′,6-diamidino-2-phenylindole (DAPI). Calbindin 2 (calretinin) is acalcium-binding protein involved in calcium signaling. Abundantly present in amacrine neurons (inner nuclear layer) and in displaced amacrine cells in retinal ganglion cell layers.

FIG. 24A and FIG. 24B are images of about 4.5 to 5-month-old retinal organoids generated from hESC lines, H1 (WA01) and ESI017. FIG. 24A shows a retinal organoid derived from the cell line, H1 (WA-01) and FIG. 24B shows a retinal organoid derived from the cell line, ESI017.

FIG. 25A and FIG. 25B are images of about 4.5 to 5-month-old retinal organoids generated from hESC lines, H1 (WA01) and ESI017. These images show the enlarged areas marked with a single (H1) and double (ESI017) asterisks to show inner- and outer segment-like protrusions emanating from the stem cell derived retinal tissue (organoids).

FIG. 26A through FIG. 26E are electron microscopy (EM) images of the rim of retinal organoids grown for about 5 months. Shown are inner segments, the connecting cilia and the short developing outer segments, similar to that of the developing dissociated and cultured photoreceptor cells.

FIG. 27A through FIG. 27F are immunohistochemical images showing expression of Rhodopsin (Rho) and Recoverin (RCVRN) in retinal organoids cultured for between about 4.5 to 5 months.

FIG. 28A through FIG. 28F are immunohistochemical images showing Rhodopsin (Rho) and Recoverin (RCVRN) staining in retinal organoids cultured for between about 4.5 and 5 months. This epifluorescent image demonstrates the distribution and the abundant presence of Rho[+] and RCVRN[+] photoreceptors in a stem cell derived retinal organoid using the methods described herein. FIG. 28C is a magnification of the retinal organoid rim, shown in FIG. 28F.

FIG. 29A and FIG. 29B are immunohistochemical images showing young developing cone photoreceptors in the about 4-month-old retinal organoid derived from human stem cells.

FIG. 30 is an immunohistochemical image showing young developing cone photoreceptors in a retinal organoid at about 4 months stained with anti-RXR gamma antibody.

FIG. 31 is an immunohistochemical image showing developing rod photoreceptors (Rhodopsin antibody) with developing outer segments, stained with Peripherin2/RDS antibody in retinal organoid at about 4.5 months.

FIG. 32 is an image of stem cell derived retinal tissue (and organoid) to be cut and transplanted into the subretinal space of a blind T-immunodeficient SD-Foxnl Tg(S334ter)3Lav (RD nude) rat.

FIG. 33 is a graph showing improvements in vison in the treatment eyes after transplantation of sections of stem cell derived retinal tissue (as measured by optokinetics) in S334ter rat.

FIG. 34 is a graph showing improvements in vison in the treatment eyes after transplantation of sections of stem cell derived retinal tissue (as measured by optokinetics) in a RSC nude rat.

FIG. 35 is an image of an optical coherence tomography (OCT) scan of the transplanted stem cell derived retinal tissue in a rat eye. The OCT demonstrates successful grafting of hESC-3D retinal tissue into the subretinal space of immunodeficient blind rats.

FIG. 36 is a fundus image that demonstrates successful grafting of hESC-3D retinal tissue into the subretinal space of immunodeficient blind rats.

FIG. 37A through FIG. 37D are images of electrode implants in blind rats to measure activation of the superior colliculus (SC) after implantation of the stem cell derived retinal tissue.

FIG. 38A through FIG. 38D are graphs showing the electrical impulses generated by activation of the superior colliculus in two disease model blind rats that were treated with stem cell derived retinal tissue implants (FIG. 38C and FIG. 38D), a sham rat (FIG. 38B) and an age matched rat control (AMC) (FIG. 38A).

FIG. 39 is a nonfluorescent immunohistochemistry image showing the grafted human stem cell derived retinal tissue implanted into the subretinal space of a disease model rat at about 6 months. Sections were stained with rabbit anti-human recoverin.

FIG. 40 is a magnified nonfluorescent immunohistochemistry image showing the grafted human stem cell derived retinal tissue implanted into the subretinal space of a disease model rat at about 6 months. Sections were stained with rabbit anti-human recoverin. Multiple rosettes of photoreceptors can be seen in the grafts with some photoreceptors forming outer segment contacts with the recipient RPE.

FIG. 41 is a nonfluorescent immunohistochemistry image showing the grafted human stem cell derived retinal tissue implanted into the subretinal space of another disease model rat at about 6 months. Sections were stained with rabbit anti-human rhodopsin.

FIG. 42A and FIG. 42B are nonfluorescent immunohistochemistry image showing the grafted human stem cell derived retinal tissue implanted into the subretinal space of a disease model rat at about 6 months, with outer segment like protrusions from Rho positive drafts extending towards the rat RPE. Sections were stained with rabbit anti-human rhodopsin. FIG. 42B is a magnification and shows integration of the graft into the rats RPE.

FIG. 43 is a nonfluorescent immunohistochemistry image showing the grafted human stem cell derived retinal tissue implanted into the subretinal space of the same disease model rat subject (rat #1704) depicted in FIG. 40 and FIG. 41, at about 6 months. Immunohistochemical analysis of human nuclei-specific antibody Ku-80 staining indicates that the graft in the subretinal space comprises human retinal tissue, and not rat retina.

FIG. 44A and FIG. 44B are fundus images of stem cell derived retinal grafts just after implantation (FIG. 44A) and at about 2.5 months after the implantation (FIG. 44B) into the subretinal space of Crx Rdy/+ cats.

FIG. 45 is an OCT image of cat eye at about 2 months and about 1 week after the implantation of the retinal tissue graft. As shown, the cat retina reattached with the RPE after implantation.

FIG. 46 is an image of a 3D reconstruction of one of the organoids in the eye shown in FIG. 45 in the cat's subretinal space, demonstrating successful grafting and reattachment of the cat retina and RPE.

FIG. 47A through 47E are a set of RetCam images showing the successful implantation of stem cell derived retinal tissue into the subretinal space of Crx+/− cat eyes. Images were taken at about 4 months after implantation.

FIG. 48A and FIG. 48B are confocal immunohistochemical images of about 6 pieces of stem cell derived retinal organoids transplanted into the subretinal space of a Crx Rdy/+ cat at about 3 months after implantation. Sections are stained with synaptophysin (SYP), recoverin (RCVRN), and DAPI.

FIG. 49A through FIG. 49C are confocal immunohistochemical images showing organoid graft/cat ONL interaction. Sections are stained with SC121, calretinin and DAPI.

FIG. 50A through FIG. 50D are confocal immunohistochemical images showing S-cone photoreceptors in the subretinal graft. Human nuclei (HNu) antibody stains human cells but not cat cells and demonstrates the differentiation between graft tissue from host tissue. Asterisks identify the area in the main image, shown in the insets. In AMD, cone regeneration or prevention of loss can improve a subject's condition because in AMD, the macula degenerates and is comprised of mostly cones.

FIG. 51 is a confocal immunohistochemical image showing human RCVRN [+] photoreceptors in the subretinal graft, cat RCVRN [+] photoreceptors in cat ONL, and human SYP[+] (human Synaptophysin) boutons in cat INL and RGC layer. This image indicates evidence of initial synaptic connectivity between the organoid graft and host. The asterisk marks the area in the main image which is enlarged in the inset. The arrows in the inset point to short inner/outer segment protrusions in rod and cone photoreceptors, organized in sheets in the cat's subretinal space.

FIG. 52 is a summary of an evaluation of human embryonic stem cell lines for differentiation into three-dimensional retinal tissue (organoids) for cell therapies of retinal degenerative conditions.

### DETAILED DESCRIPTION

Stem cell derived retinal tissue described herein may be used to provide sustained neurotrophic support to degenerating retinal tissue in a subject.

In some aspects, cell therapy compositions are described which provide a combination approach of delivering a cocktail of neuroprotective factors simultaneously from the vitreous side and in direct proximity to a subject's degenerating retinal tissue. This approach can provide long-lasting neuroprotection in subjects with retinal degenerative diseases, disorders or trauma related retinal damage or degeneration.

Sustained localized intra-ocular and intra-retinal release of trophic factors (e.g., BDNF, NGF) and/or mitogens (e.g., bFGF) and/or, neuroprotective exosomes carrying microRNAs, or their combination) from integrated epiretinal grafts of retinal progenitor cells isolated from stem cell derived retinal tissue that migrate into the recipient's retina from these grafts can provide a continuous therapeutic dosage of molecular trophic support.

The terms “stem cell derived retinal tissue” “hESC-derived 3D retinal tissue”, “human pluripotent stem cell (PSC)-derived retinal tissue”, “hESC-derived 3D retinal organoids”, “hPSC-derived retinal organoid”, “hESC-3D retinal tissue,” “in vitro retinal tissue,” “retinal organoids,” “retinal spheroids” and “hESC-3D retinal organoids” are used interchangeably in the present disclosure and refer to pluripotent stem cell-derived three-dimensional aggregates comprising retinal tissue. The stem cell derived retinal tissue develops retinal layers (e.g., RPE, PRs, inner retinal neurons (i.e., inner nuclear layer) and retinal ganglion cells), also Muller glia cells, and display synaptogenesis and axonogenesis commencing as early as around 6-8 weeks in certain organoids and can become more pronounced at around 3^{rd} or 4^{th} month of hESC-3D retinal development. The stem cell derived retinal tissue may be genetically engineered to transiently or stably express or overexpress a transgene of interest or not express certain human gene (via gene silencing or gene knockout) or express genes at lower levels than in normal developing retinal tissue to achieve retinal tissue compatibility with the recipient and/or to modify the differentiation fate of retinal cells in the hESC-derived retinal organoids, e.g., to enhance photoreceptor differentiation or rod versus cone cell fate determination or/and to suppress certain cell fates in developing hESC-derived retinal organoids). Stem cells, including human embryonic stem cells (hESCs) and human pluripotent stem cells in general, provide a reliable source for cell therapies.

Although the present disclosure refers to hESC-derived 3D retinal tissue, it will be appreciated by those skilled in the art that any pluripotent cell (ES cell, iPS cell, pPS cell, ES cell derived from parthenotes, and the like), may be used as a source of 3D retinal tissue according to methods of the present disclosure.

As used herein, “embryonic stem cell” (ES) refers to a pluripotent stem cell that is 1) derived from a blastocyst before substantial differentiation of the cells into the three germ layers; or 2) alternatively obtained from an established cell line. Except when explicitly required otherwise, the term includes primary tissue and established cell lines that bear phenotypic characteristics of ES cells, and progeny of such lines that have the pluripotent phenotype. The ES cell may be human ES cells (hES). Prototype hES cells are described by Thomson et al. (Science 282:1145 (1998); and U.S. Pat. No. 6,200,806), and may be obtained from any one of number of established stem cell banks such as UK Stem Cell Bank (Hertfordshire, England) and the National Stem Cell Bank (Madison, Wis., United States). Example cells line include but are not limited to H1 (WA01) and HAD-102.

As used herein, “primate pluripotent stem cells” (pPS) refers to cells that may be derived from any source and that are capable, under appropriate conditions, of producing primate progeny of different cell types that are derivatives of all of the 3 germinal layers (endoderm, mesoderm, and ectoderm). pPS cells may have the ability to form a teratoma in 8-12 week old SCID mice and/or the ability to form identifiable cells of all three germ layers in tissue culture. Included in the definition of primate pluripotent stem cells are embryonic cells of various types including human embryonic stem (hES) cells, (see, e.g., Thomson et al. (1998) Science 282:1145) and human embryonic germ (hEG) cells (see, e.g., Shamblott et al., (1998) Proc. Natl. Acad. Sci. USA 95:13726,); embryonic stem cells from other primates, such as Rhesus stem cells (see, e.g., Thomson et al., (1995) Proc. Natl. Acad. Sci. USA 92:7844), marmoset stem cells (see, e.g., (1996) Thomson et al., Biol. Reprod. 55:254,), stem cells created by nuclear transfer technology (U.S. Patent Application Publication No. 2002/0046410), as well as induced pluripotent stem cells (see, e.g., Yu et al., (2007) Science 318:5858); Takahashi et al., (2007) Cell 131(5):861). The pPS cells may be established as cell lines, thus providing a continual source of pPS cells.

As used herein, “induced pluripotent stem cells” (iPS) refers to embryonic-like stem cells obtained by de-differentiation of adult somatic cells. iPS cells are pluripotent (i.e., capable of differentiating into at least one cell type found in each of the three embryonic germ layers). Such cells can be obtained from a differentiated tissue (e.g., a somatic tissue such as skin) and undergo de-differentiation by genetic manipulation which re-programs the cell to acquire embryonic stem cell characteristics. Induced pluripotent stem cells can be obtained by inducing the expression of Oct-4, Sox2, Kfl4 and c-Myc in a somatic stem cell. Thus, iPS cells can be generated by retroviral transduction of somatic cells such as fibroblasts, hepatocytes, gastric epithelial cells with transcription factors such as Oct-3/4, Sox2, c-Myc, and KLF4. Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); 111 Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872. Other embryonic-like stem cells can be generated by nuclear transfer to oocytes, fusion with embryonic stem cells or nuclear transfer into zygotes if the recipient cells are arrested in mitosis.

It will be appreciated that embryonic stem cells (such as hES cells), embryonic-like stem cells (such as iPS cells) and pPS cells as defined infra may all be used according to the methods of the present invention. Specifically, it will be appreciated that the hESC-derived 3D retinal organoids/retinal tissue may be derived from any type of pluripotent cells.

Retinal tissue derived from human embryonic stem cells have been shown to recapitulate the anatomical structure, biological complexity and physiology of developing human retinal tissue and have all retinal layers (PRs, 2^{nd} order neurons, retinal ganglion cells) and RPE from hESCs. Human stem cell derived retinal tissue has also been shown to display characteristics very similar to human fetal retina at early developmental stages (week 8-16), display robust synaptogenesis and electrical activity after 8 weeks of development, and contain rudimentary inner segment-like protrusions immunopositive for peanut agglutinin (PNA).

New methods of deriving retinal tissues from stem cells are presented herein and include the use of wheat germ agglutinin (WGA). These methods enable large scale simplified production of stem cell derived retinal tissue useful for treating retinal degenerative diseases and disorders. Hundreds of stem cell derived tissue aggregates (or organoids) can be generated from any ES or iPS cells of human primate, canine and feline origin growing in adherent conditions or in suspension starting from 10×100 mm plates with predictable characteristics (RPE, PR, RGC layers) and a predictable derivation timeframe. In some aspects, cells are produced in a bioreactor.

Stem cell derived retinal tissue can be transplanted as tissue aggregates or can be dissociated to single cells or clumps of cells to generate retinal progenitor cells for transplantation. These cells may be administered in suspension in a pharmaceutically acceptable carrier or combined with a biomaterial.

In some aspects, stem cell retinal tissue (organoids) can be administered as a bioprosthetic patch or implant. The organoids can be combined with or attached to or embedded within a biocompatible material to generate a retinal patch or implant. In some aspects, stem cell derived retinal tissue may be transplanted as sheets of photoreceptors.

Stem cell derived retinal tissue compositions are stable and can be shipped at 37° C. overnight.

Retinal remodeling is a secondary cause of vision loss in retinal degeneration and preventing remodeling can be an aspect of neuroprotective therapy. Transplanted cells and/or tissue can be used as mini-factories which produce trophic and other neuroprotective factors over an extended period of time. Transplanted retinal progenitor cells isolated from stem cell derived retinal tissue can stay in the epiretinal grafts (where they stay at the same level of differentiation or undergo differentiation) and/or migrate into the recipient retina, differentiate into the postmitotic region-specific retinal cells and integrate into the neural architecture of the recipient retina structurally and/or synaptically.

Neurotrophic factors include a diverse group of soluble proteins (neurotrophins), and neuropoietic cytokines, which support the growth, survival and function of neurons. They can activate multiple pathways in neurons, ameliorate neural degeneration, preserve synaptic connectivity and suppress cell death in retinal tissues. Acutely injured retina may survive if neuroprotection, provided in the form of small molecules, neuroprotective proteins such as Brain-Derived Neurotrophic Factor (BDNF), or cells, is delivered early enough to suppress cell death and/or initiation of retinal remodeling and scarring.

In addition to neurotrophic factors and neuropoetic cytokines, other components of the cellular secretome may be useful in preserving and/or regenerating retinal tissue. Exosomes are small vesicles of endosomal origin, which are secreted by cells, and carry proteins, RNA, long non-coding RNAs (1nRNAs) and especially microRNAs. MicroRNAs themselves may be released from various cells and are used for paracrine interaction. Neuropeptides are classical hormones, used for extracellular communication, including neuroendocrine cells, which may work via paracrine mechanism or/and via blood stream release. Neurophospholipids/fatty acids are also part of cellular secretome and can be neuroprotective. These factors can be used to provide a neuroprotective effect exerted by transplanted cells. Preferably, therapeutic compositions continuously deliver a steady flow of a neuroprotective cocktail via a localized paracrine mechanism to ensure a continuous and effective dosage of neuroprotectants.

Selected trophic factors (TFs), such as brain-derived, glial-derived neurotropic factors (BDNF, GDNF) and nerve growth factor (NGF), can exert a powerful neuroprotective effect on mammalian retina in vivo, attenuate the PR cell death and transiently ameliorate blindness. Most of these TFs have evolved to cause a very localized, yet steady paracrine or even autocrine positive effect during the Central Nervous System (CNS) development, including retinogenesis.

In some aspects, a high and sustained level of neuroprotection is delivered into degenerating retinas by embedding or transplanting tropic factor and/or other neuroprotective factor-expressing retinal cells into the ocular (e.g., epiretinal or vitreous or subretinal) space. Grafted cells may integrate into the neural architecture of degenerating retina, thus strengthening it and slowing retinal remodeling. In some aspects, the cells may be genetically altered to express or overexpress certain neuroprotective factors. In some aspects, these trophic factors may be selected for their ability to support PRs in a degenerating retina and to promote synaptogenesis and axonogenesis. Examples of genes that may be overexpressed include OPN, IL6, VEGFA, CXCL12, PTN, Lefty2, FGF9, ctgf, JAG1, NOG, KDR, Nodal, NRG1, hbergf, bmp2, ngfr, gdf11, tgfb1, MDK, cxcr4, sod1, B2M, SDF, CRABP1, SIRT2, SERPINF1, CLU, and BSG. In some aspects, the retinal cells delivered as epiretinal grafts, may become an integral part of the recipient neural circuitry, thus combining neuroprotection and cell replacement.

Neuroprotective factors include proteins and other molecules that promote the proliferation, differentiation, and functioning of neurons and other cells, and protect from apoptosis. Neurotrophic factors may include but are not limited to, trophic factors, mitogens, microRNAs, exosomes, or their combination. Delivering neuroprotective factors to degenerating retina from the epiretinal grafts via paracrine mechanisms can eliminate the retinal trauma to fragile and degenerating retinal tissue, attenuate vision loss in RD retina and even improve vision. Sustained localized intra-ocular and intra-retinal release of trophic factors (e.g., BDNF, NGF) or/and mitogens (e.g., bFGF) or/and, neuroprotective exosomes carrying microRNAs, or their combination from integrated retinal progenitor cells from stem derived retinal tissue can provide a continuous therapeutic dosage of molecular trophic support, ameliorating and/or preventing additional vision loss in subjects with degenerative retinal conditions.

In some aspects, 11-cis retinal may be administered to aid in neuroprotection and cell support. 11-cis retinal is normally produced by the RPE.

In some aspects, transplantation of retinal progenitor cells dissociated from stem cell derived retinal tissue may be combined with gene therapies and cell replacement therapies.

Conditions in which the compositions described herein are useful for treating include, but are not limited to, Age-related macular degeneration (AMD), geographic atrophy, retinitis pigmentosa, Leber congenital amaurosis, diabetic retinopathy, retinopathy of prematurity, ocular trauma-related retinal injuries, glaucoma, retinal degenerative disease, intermediate dry AMD, retinal detachment, retinal dysplasia, retinal atrophy, retinopathy, macular dystrophy, cone dystrophy, cone-rod dystrophy, Malattia Leventinese, Doyne honeycomb dystrophy, Sorsby's dystrophy, pattern/butterfly dystrophies, Best vitelliform dystrophy, North Carolina dystrophy, central areolar choroidal dystrophy, angioid streaks, toxic maculopathy, Stargardt disease, pathologic myopia, and macular degeneration.

In some aspects, administration of the stem cell derived retinal tissue and/or retinal progenitor cells dissociated from stem cell derived retinal tissue includes but is not limited to epiretinal, vitreal injections. Tissue or cells may be administered into the vitreous above the degenerating retinal area. In some aspects, delivery of retinal tissue or cells is non-invasive or minimally invasive. In other aspects, administration of retinal cells dissociated from stem cell derived retinal tissue does not cause epiretinal membranes or retinal detachment. Epiretinal grafting is not damaging to an already degenerating and very sensitive neural retina. Implanted progenitor cells do not block vision due to the transparency of the cells (no pigment).

In some aspects, the graft (stem cell derived retinal tissue or organoid) is placed close to the RPE of the recipient and form a sandwich between the recipient's RPE and the degenerating neural retina. In some aspects, the implantation only produces a small injury to the retina, with use of a small-sized retinotomy. Additionally, the graft is retained within the subretinal space.

In an aspect, provided herein are a method of generating retinal progenitor cells, the method including differentiating stem cells into retinal tissue in a medium comprising lectin; and dissociating the retinal tissue to isolate retinal progenitor cells.

In embodiments, differentiating stem cells includes (i) obtaining pluripotent stem cells; (ii) culturing pluripotent stem cells in mTESR media for about 5 to about 8 days; and (iii) further culturing the pluripotent stem from about day 5 or about day 8 until about day 30 in a medium comprising lectin until retinal tissue is formed.

In embodiments, dissociating retinal tissue to isolate retinal progenitor cells includes harvesting organoids by digesting with enzyme. In embodiment, the enzyme is papain.

According to some embodiments, the methods described herein further comprise, administering immunosuppression to the subject for one day to three months after the administration of retinal progenitor cells or tissue grafts. According to other embodiments, the methods described herein further comprise, administering immunosuppression to the subject for three months after the administration of retinal progenitor cells or tissue grafts. According to other embodiments immunosuppression is not provided.

### EXAMPLES

The following examples are put forth so as to provide those of ordinary skill in the art with a complete disclosure and description of how to make and use the present methods and compositions and are not intended to limit the scope of what the inventors regard as their disclosure nor are they intended to represent that the experiments below are all or the only experiments performed.

### Example 1

Derivation of Human Retinal Tissue from Human Pluripotent Stem Cells Using Lectin Wheat Germ Agglutinin (WGA)

Human pluripotent stem cells were cultured on Matrigel coated plates (or vitronectin or laminin-521 or growth factor reduced Matrigel or other suitable substrate that will maintain stem cell pluripotency) until colonies reached ˜1-2 mm size in diameter or more (about 5-8 days) in mTESR-1 media. For about 1-7 days, the media was not changed.

On about day 8, the mTESR1 media was changed to 1:1 mTESR1 and Neurobasal complete medium. Neurobasal medium, 94.8%, 1×N2, 1×B27 without retinoic acid (ThermoFisher), Pen/Sterp antibiotic (1% vol/vol), 1-glutamine (1% vol/vol), 1% Minimal Essential Medium nonessential amino acid solution (MEM vol/vol), 1× amphotericin-B/gentamicin (ThermoFisher), BSA fraction V (0.1%) (Sigma-Aldrich), b-mercaptoethanol (0.1 mM; Sigma-Aldrich). The stem cells were cultured from about day 8 to about day 30 in the neurobasal media. Half of the media was changed about every three days.

Alternatively, the cells may be cultured in BrainPhys-embryonic complete (BrainPhys media-94.8%, Stem Cell Technologies, SM1 without retinoic acid 1× (Stem Cell Technologies), N2-embryonic 1× (Stem Cell Technologies), BSA 0.1% (Sigma Aldrich, Fraction V), Pen/Strep 1×, L-Glutamin 1×, Non-Essential amino Acids 1×, Gentamycin/Amphotericin 1× (ThermoFisher), beta-mercaptoethanol 0.1 mM, (Sigma-Aldrich) or Neurobasal complete Composition. Neurobasal medium, 94.8%, 1×N2, 1×B27 without retinoic acid (all 3 from ThermoFisher), Pen/Sterp antibiotic (1% vol/vol), 1-glutamine (1% vol/vol), 1% Minimal Essential Medium nonessential amino acid solution (MEM vol/vol), lx amphotericin-B/gentamicin, BSA fraction V (0.1%) (Sigma-Aldrich), b-mercaptoethanol (0.1 mM; Sigma-Aldrich).

Wheat Germ Agglutinin (WGA (lectin)) (at a concentration of between about 0.5 μg/ml to 0.5 mg/ml, preferably at a concentration of about 5 μg/ml) and human recombinant noggin (at a concentration of between about 1 ng/ml to about 250 ng/ml, and preferably at a concentration of about 50 ng/ml, R&D systems or other source) were added to the cells for between about 4-7 days at about 37° C., normoxia, about 5% CO2. Between about 3-20% oxygen and between about 5-10% CO2 may be used. Optionally, WGA may be used alone to generate stem cell derived retinal tissue. Also, DAPT may be added to the differentiation media.

After removing lectin by changing the media, differentiating cells were feed by replacing half of the media with BrainPhys-embryonic complete or Neurobasal complete or a combination of media compositions at between about 1:99 to 99:1 (e.g., 50% BrainPhys complete and 50% Neurobasal complete) without FBS. Cells were kept at 37° C., normoxia, 5% CO2. WGA lectin application and/or WGA lectin+noggin application leads to the downregulation of TGF, BMP, FGF, NODAL, WNT pathways.

After about 3-6 weeks, foci of differentiation were cut out from dishes manually using a sharp sterile tool and cultured in nonadherent conditions using a shaker (about 30-50 rpm) using the same media+Taurine (at about 100 μM), 10% Fetal Calf Serum (FCS, DHA are optional). Adding basic FGF at a concentration of between about 0.01 to about 100 ng/ml, preferably 20 ng/ml and BDNF (at a concentration of about 20 ng/ml) is optional but can help promote growth. Optionally, all-trans Retinoic acid (RA) (at a concentration of between about 0.01 to about 5 μM, but preferably at a concentration of about 0.5 μM) may be added to the culture alone or in combination with either 2 or 3 (bFGF, BDNF, RA).

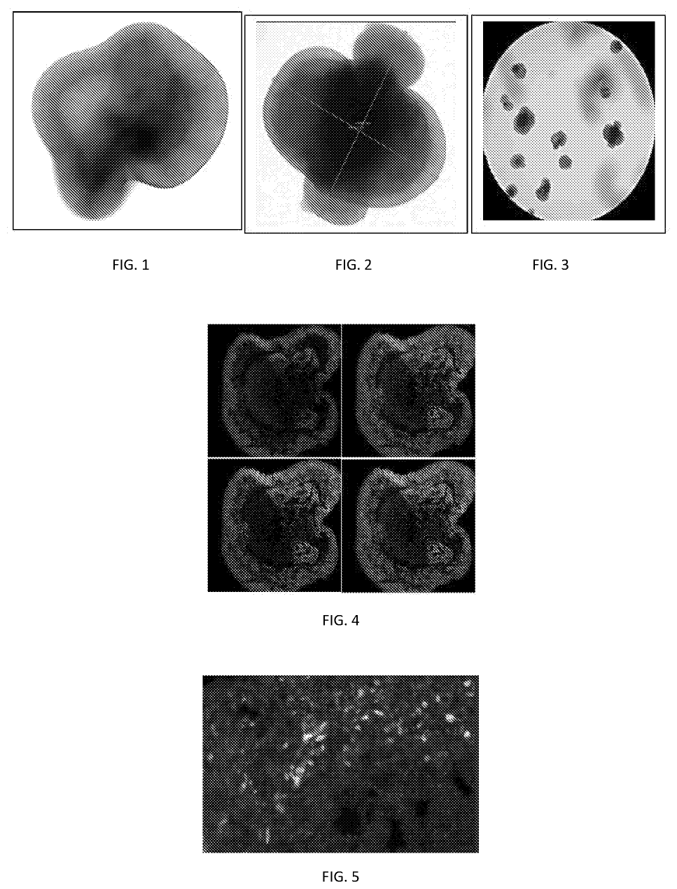

Stem cell derived retinal tissue may be maintained in static conditions and cultured, with all-trans retinoic acid added at between about day 40-50 after initiation of differentiation, in single 96-wells of a 96-well ultra-low adhesion plates or other substrates/materials which do not promote adhesion. Such conditions prevent retinal tissue from adhering to each other and promotes maturation, lamination and formation of inner and outer segments. FIG. 1 shows an image of a developing stem cell derived retinal tissue aggregate (organoid) at month 4-5. FIG. 2 shows an image of developing stem cell derived retinal tissue aggregates (organoids) at month 4-5 at a size of about 1.6 mm by about 1.62 mm. FIG. 3 shows an image of developing stem cell derived retinal tissue aggregates (organoids) at month 4-5.

Retinal tissue derived from stem cells using lectin WGA (or lectin WGA+noggin) have a rim with cells positive for PAX6, CHX10, VSX2, (neural retina marker), many cells are BLIMP1 [+] (photoreceptor progenitor marker), BRN3A/B/ISL1/TUJ1 (markers of retinal ganglion cells), Calretinin and Calreticulin (marker of amacrine neurons), by between about week 10-12, and many recoverin [+] Trbeta2[+], RXRgamma [+] (or any combination thereof) by between about the 12 to 14 week of differentiation.

FIG. 4 shows images of an immunohistochemical assay. Stem cell derived retinal tissue derived from the H1 human ESC line is shown positive for CHX10 and PAX6, markers of developing neural retina, at between 2 and 3 months after initiating differentiation. Retinal organoids derived from other lines demonstrated the same distribution of these markers (not shown).

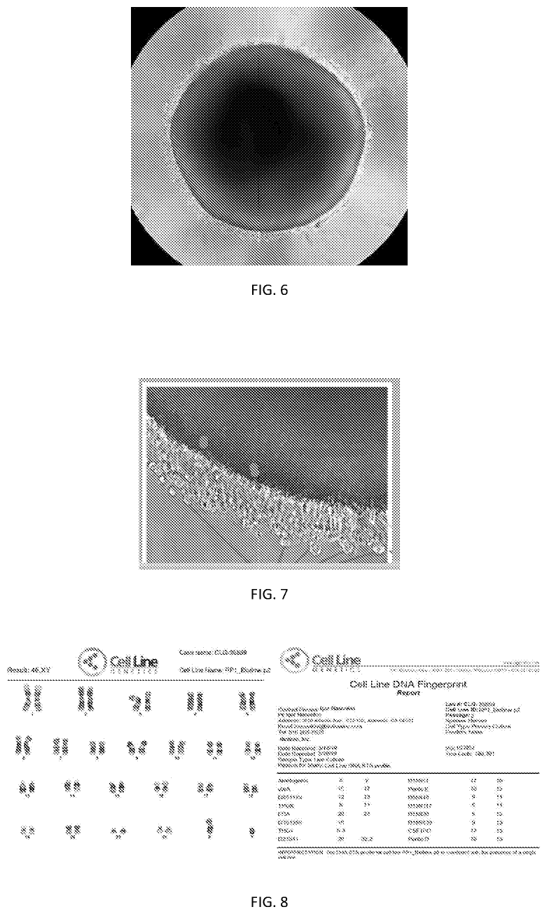

FIG. 5 shows an immunocytographic image of the rim of the stem cell derived retinal tissue. Cells are shown staining positive for Recoverin and Trβ2, illustrating the developing cone photoreceptors. Cell nuclei are stained with DAPI. FIG. 6 shows a magnified image of stem cell differentiated tissue, with the dark pigmented cells in the center and the visible outer rim protrusions. FIG. 7 shows the beginning of developing inner and outer segments with the cilia in photoreceptors within the rim about 4-5 months after differentiation using lectin. These organoids have prominent Rhodopsin staining (marker of rod photoreceptors) and Recoverin (marker of rod and cone photoreceptors and cone bipolar cells) in the rim and outer segment-like protrusions.

Transcriptome analysis (RNA Seq) of the stem cell derived retinal tissue was performed by BGI Genomic Services (Cambridge, Mass.). Genes OPN, IL6, VEGFA, CXCL12, PTN, Lefty2, FGF9, ctgf, JAG1, NOG, KDR, Nodal, NRG1, hbergf, bmp2, ngfr, gdf11, tgfb1, MDK, cxcr4, sod1, B2M, SDF, CRABP1, SIRT2, SERPINF1, CLU, and BSG were found to be upregulated in stem cell derived retinal tissue differentiated using lectin WGA. This large number of transcripts of proteins, which are expected to be secreted (exported) by the cells that make up the stem cell derived retinal tissue and may contribute to neuroprotective qualities of cell preparations from retinal organoids.

### Example 2

### Retinal Progenitor Cells Isolated (Dissociated) from Stem Cell Derived Retinal Tissue

Organoids were harvested at about day 49-70 to yield between about 0.3-0.8 mm or larger diameters using a papain kit from Worthington biochemicals by digesting with papain for preferentially about 20 min at about 37° C. The culture was spun, the supernatant removed, and about 10 ml of media comprising neurobasal complete, described above at about 60% per volume, mTeSR-1 complete, about 20% per volume, BrainPhys complete with N2-embryonic, lx, SM1, lx supplements at about 20%, 0.5× Rock inhibitor (5 μg/ml), optionally 0.5× Nicotinamide (vitamin B3 or NIC) (at a concentration of about 1-50 but preferably 5 amphotericine/gentamicin 1×, 20 ng/ml each basic FGF (bFGF) (RnD systems or other supplier), BDNF, optionally epithelial growth factor, EGF) was added.

Cells were plated on either fresh GFR Matrigel plates (prepared as: 500 μl ice-cold Matrigel/50 ml ice-cold DMEM media, with 5 ml mix for each 10 cm tissue culture plate, until solidified—about 1 hour) or vitronectin coated plates or gelatin coated or Lam521 coated pates or any other laminins or their combinations, or human fibronectin coated plates or polyornithine (PORN)-coated or hydrogel coated or any other substrate appropriate for cell culture. Plates were left unchanged for about 2 days, incubated in a humidified tissue culture incubator at either low oxygen (3-5% oxygen hypoxic conditions), or mild hypoxia (between 5 and 20% oxygen), or normoxia (21%) or hyperoxia (above 21%), and CO2 5-10%. Half of the media was changed on day 3 with same media or media comprised of one or more of: neurobasal complete, brainphys complete, and mTeSR1, each varying from 100% to 0%, without fetal bovine or any other kind of serum. Papain or manual passaging were used as preferred methods or alternatively, other enzymes e.g., trypsin-like enzyme, and accutase may be used. Cells were digested into small clumps for passaging. Stocks were frozen at passages P1, P2, P3.

As an option: before dissociating stem cell derived retinal tissue with papain, the rims of the organoids can be cut with fine vitreoretinal or ophthalmic scissors or a fine surgical scalpel, and dissociated. As another option: dissociate whole organoids or precut rims of organoids, sort for c-kit (young progenitors) or CD-73 (photoreceptors) or CD24 or CD-133 or CD-15 or with antibodies for any other surface determinant (CD) marker present in human developing retina, select cells (or, select out cells) and culture separated cells as above. Also optionally, DAPT may be added to the cell culture medium before retinal progenitor cells are transplanted to slow cell division.

### Example 3

Karyotyping and Fingerprinting

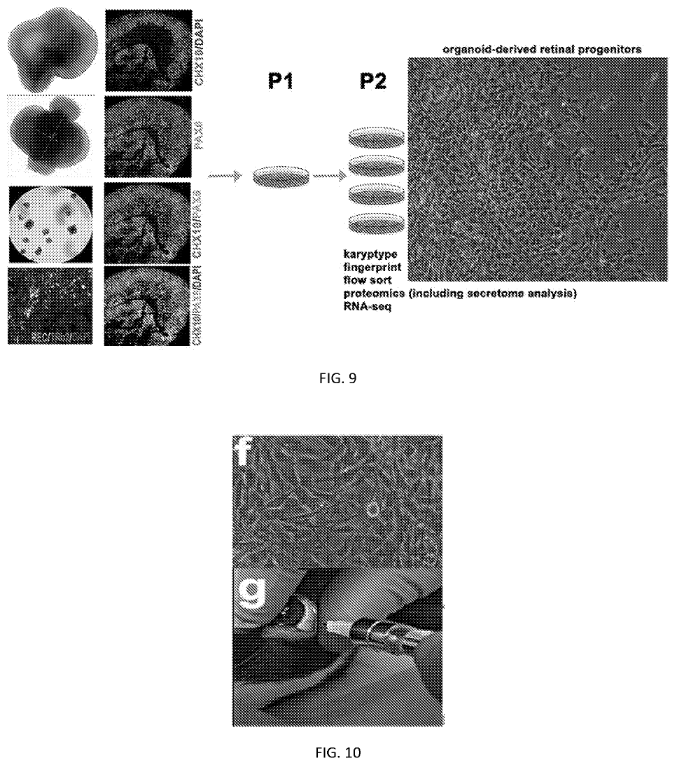

Retinal progenitors (Passage 2, (P2)) were karyotyped and DNA-fingerprinted by CellLine Genetics. Detailed microdeletion/microduplication analysis (with 1 MB or more resolution) was carried out by Life Technologies cytogenetic services. Karyotype of P2 retinal progenitors was normal, with no deletions/duplications and no trisomies or/and translocations, as shown in FIG. 8. The DNA fingerprinting signature matched the parental H1 (WA01) hESC line, was consistent with the presence of a single cell line (without admixture from other lines) and showed XY chromosomes as H1 line is a male line. The analysis demonstrates that our differentiation and passaging procedures do not cause chromosomal aberrations (which may be a contributing factor for tumorigenesis in grafts). In addition to karyotyping, proteomics/secretome analysis and cell sorting data may be obtained, also shown in FIG. 9.

### Example 4

Young progenitor cells or semi-differentiated cells are capable of secreting neuroprotective factors and may deliver a steady level of neuroprotection from the vitreous side via paracrine secretion after being incorporated into the recipient's neural retinal layers (e.g., RCG, INL, etc.). The integrated cells can strengthen the architecture of degenerating retina, thus ameliorating vision loss.

Cultures of human embryonic stem cell derived retinal tissue retinal progenitor cells were grafted in immunosuppressed (Cyclosporin A+prednisolone) adult rabbits without retinal degeneration (3 rabbits/each cell dosage). Cell doses comprised 0.5×10⁶, 1×10⁶ and 1.5×10⁶ human embryonic stem cell derived retinal tissue retinal progenitor cells per eye. Vitreal injections comprising saline were first injected to determine the surgical procedure used for injecting cells.

Human embryonic stem cell derived retinal tissue retinal progenitor cells at passage 2 were slowly injected using a 50 μl Hamilton syringe with a luer-lock, and 27 g needle inserted into the pars plana, into the vitreous space, above the RGC layer, as shown in FIG. 10. Several rabbits were assigned to the control group (sham-injected with α-cellular preparation of conditioned medium from retinal progenitors). The treated eyes were continuously observed and OCT (also assayed prior to treatment), B-scan ultrasonography, fundus imaging and electroretinography (ERG).

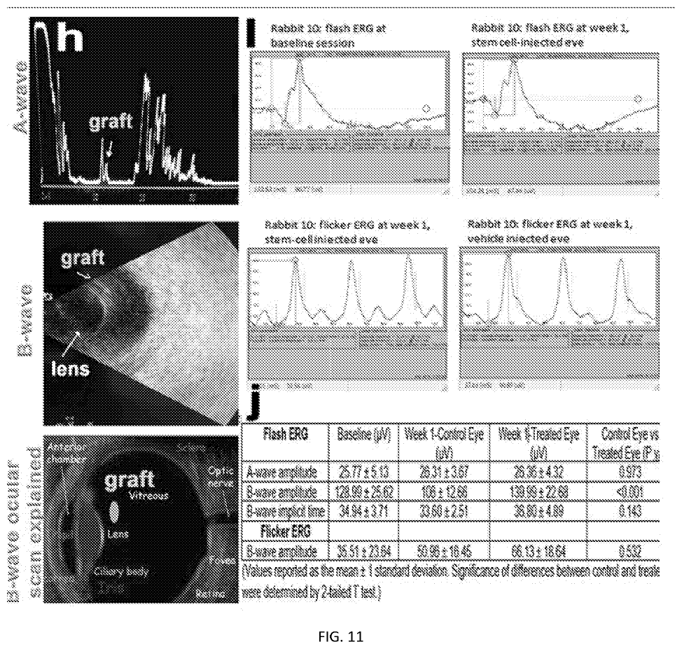

All rabbits demonstrated normal ERG (no decrement in ERG signal), no retinal thickening, no external inflammation of the eye, normal fundus and no signs of distress at about 1.5 months after transplantation (and grafting) of cells. While this OCT assay was unable to detect the epiretinal grafts (likely due to their low density), the A and B-scan ultrasonography was able to detect cells above the retina as shown in FIG. 11. A and B-wave ocular ultrasonography data (left panels of FIG. 11) performed on a rabbit eye given a dose of 0.5×10⁶ retinal progenitors grafted into the vitreous space are presented. The white arrows show a signal from the grafted cells. The lower panel shows the B-wave to outlining the shapes of the major anatomical structures within the eye. The graphs show flash and flicker ERG responses recorded from a rabbit with retinal progenitor graft in the vitreous. Control=untreated eye (vehicle=conditioned medium). The signals are almost identical, demonstrating that organoid-derived retinal progenitors do not cause acute adverse reaction in the recipient large eye model. The table in FIG. 11 shows little difference in a, b-wave amplitude and flicker ERG between control (n=3) & treated (n=9) eyes.

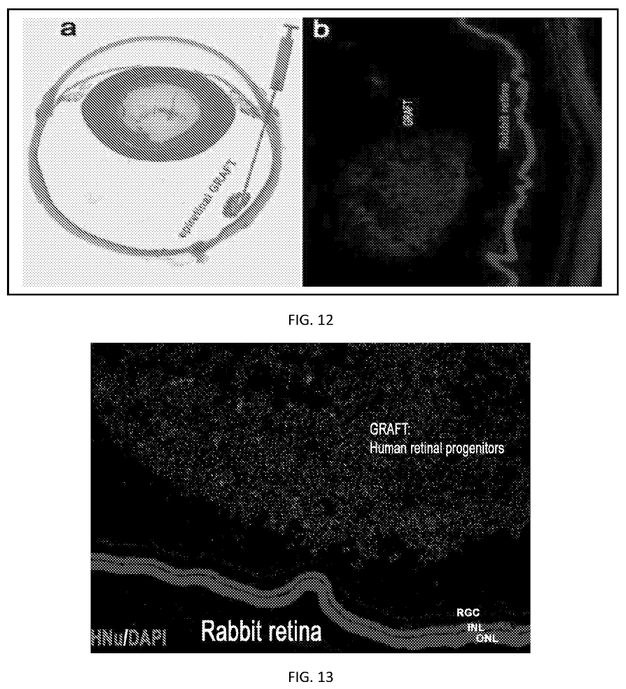

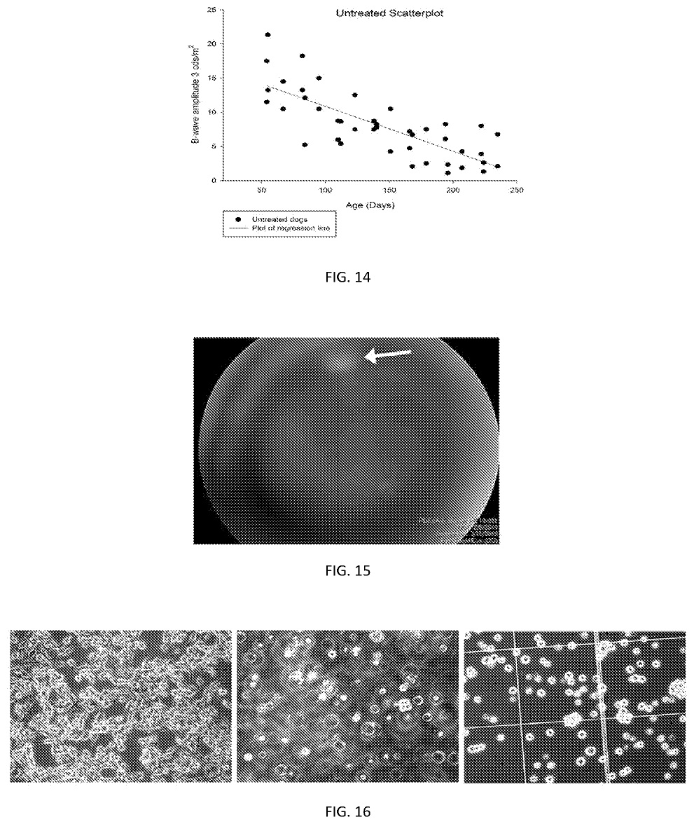

Imaging of delivered retinal progenitors into rabbit eyes demonstrate that the cells can be successfully delivered into a large eye, and are not dispersed in the vitreous, but rather, remain as a bolus. No to minimal fluid reflux was observed, which may be associated with the loss of injected cells. Is expected that the epiretinal grafts may migrate within the eye and may be found in different areas within the vitreous. FIG. 12 shows ex vivo delivery of 3×10⁶ organoid-derived human retinal progenitors into a rabbit eye after euthanization and removal of the eye. The eye had normal intra ocular pressure and there was no fluid reflux observed. FIG. 13 shows an image of immunostatined human retinal progenitor cells after delivery into a rabbit eye ex vivo.

### Example 5

To analyze the effect of human embryonic stem cell derived retinal tissue retinal progenitor cells on providing retinal neuroprotection, dog model of early-onset RP was used. Epiretinal grafting of human embryonic stem cell derived retinal tissue retinal progenitor cells was performed on 2 PDE6A^{-/-} dogs (Cardigan Welsh corgi breed), age 4 weeks. Retinal degeneration follows a classical rod-cone dysplasia (RCD) model, where rods (PDE6A gene is expressed) die first, followed by cone degeneration (as a consequence of rod degeneration). Preserving rod PRs enables cone preservation as well. Functional vision testing has been worked out in dogs, enabling testing of the efficacy of such therapy. The dynamics of RD were delineated in this model and it was determined that therapeutic intervention (such as neuroprotection) can be administered early, before RD and gliosis takes place. Gradual loss of cones appears between 1-7 months, as shown in FIG. K. FIG. K shows the gradual decline in cone ERG between about day 50 and 150 in a large cohort of PDE6A^{-/-} dogs.

Harvested cells were injected at a dose of 1.7×10⁶ human retinal progenitors into OD of two PDE6A^{-/-} dogs. Fundus examination was done using RetCam imaging equipment and demonstrated a graft located close to the peripheral retina, as shown in FIG. 15. Post-op cell viability (Trypan Blue, 99.5% viability) and cell vitality tests (by plating 1, 2 and 5 microliters of cells in the remaining preparation after injecting of the 2^{nd} dog; 95%+ of cells attached within 1h) were performed. The dogs were followed with weekly ERG and RetCam imaging tests, which revealed no ocular abnormalities and no impact on PR function. FIG. 16 shows human embryonic stem cell derived retinal tissue retinal progenitor cells at passage 2, dissociated with papain.

The neuroprotective impact of dissociated retinal progenitor cells from human embryonic stem cell derived retinal tissue can be assessed using sensitive electrophysiological techniques (visually evoked potential, ERG) coupled with ocular imaging, full field ERG (Espion II unit from Diagnosys LLC), RETImap system for multifocal ERG, OCT, RetCam imaging and the behavioral method for objective vision testing (an obstacle course for dogs; optokinetic tracking system for cats), and Visual Evoked Potentials (VEPs).

### Example 6

Dose preparation efficacy of retinal progenitor cells will be determined. A 1-2-3 passage of primary neuronal cultures dissociated from tissue will be established and expanded, can be easily cryopreserved, and has the potential to produce improved results after grafting. Low passage of cells (e.g., P2) may help to safeguard against inducing chromosomal abnormalities. Different lines of hESCs may differ in the ability to differentiate into various cell lineages and types due to slightly different epigenetic marks and genetics (combination of different alleles) and progeny of the same lineage (e.g., retinal) may have slightly different transcriptome, impacting neuroprotective qualities of cells. Retinal progenitors that do not mature in the epiretinal space, may enhance neuroprotective efficacy of the epiretinal grafts.

RNA-Seq profiling of hESC-3D retinal tissue (retinal organoids) from several different hESC lines (each with a stable and normal karyotypes) will be conducted and the level of potentially neuroprotective transcripts in these lines will be compared. As a control, we will use transcriptome from week 11-week 16 human fetal retina samples.

It is acknowledged that neuroprotective qualities of cells may not be based only on the expression of NGF, BDNF or/and other known neuroprotective factors but/or/in addition, presence of less known factors or/and microRNAs, exosomes, neuropeptides, neurolipids etc. Therefore, a pilot evaluation of a passage (P)2 organoid-derived retinal progenitors will be conducted by transplanting retinal cells from human embryonic stem cell derived tissue into the vitreous space of RCS rats. The RCS rat is a model of RD that is widely-used. By 100 days, less than 20% of PRs remain in the ONL of RCS rats due to MerTk mutation in RPE. We will transplant 60,000 retinal progenitors/OD eye at the age of 20 days (before the onset of RD), and do sham grafting (conditioned medium only) in counterpart (OS) eye, wait 3 months, evaluate visual function by RGG and optokinetic testing, and dynamics of retinal degeneration by OCT, sacrifice the animals at 3 months, perform histology/IHC analysis, determine, compare and quantify preservation of retinal thickness.

The retinal explant model may not take into the account the impact of the immune system, immunosuppression, cell dosage/eye, potential of grafts to over-proliferate & other adverse graft-host retina interactions, the ocular pressure and surgical delivery (which influence graft distribution), and retinal physiology (critically affecting the visual function). The explant model may be an auxiliary rapid test and can be combined with robust in vivo assay demonstrating lack of adverse reaction to the ocular tissue and vision in general.

While an in vivo test in the RCS rats will be our primary method of choice, we will nevertheless evaluate an auxiliary ex vivo assay of testing batches of cells for neuroprotection using whole eye (rodent, rabbit) cultures. We will use mouse eyes as a primary ex vivo model, and test (i) normal postnatal (P)15 eyes and (ii) P15 eyes of rd10^{Pde6b-/-} mice. We will isolate the eyecups with long optic nerves, making sure not to cause retinal detachment, remove corneas, graft 60,000 cells/eyecup (experimental group) or conditioned medium (controls), in the volume of 2 microliters, and culture the eyecups in either normoxic or hypoxic conditions in the media. The paracrine flow of neuroprotective secretome from the grafts can ameliorate the deterioration of neural retina-RPE-Bruch's membrane layers and this will be our quick readout (ex vivo efficacy assay). We will initially culture the eyecups for 10 days, fix in 4% paraformaldehyde solution, generate frozen sections and evaluate the histological preservation of the eyecups, with focus on the ONL thickness and the OS-RPE junction. We will then use markers of cell death such as Cleaved Caspase-3 and [symbol] H2AX. Preservation of the eyecups in these conditions will be measured, and improved preservation in the group, grafted with neuroprotective retinal progenitors. The culture time may be extended for 2-3 weeks.

Transcriptome, proteome and secretome analysis (also mass spec) will be conducted on retinal progenitors and their conditioned medium. At least 3 sources of stem cells will be evaluated for efficacy of RPE cell replacement. In addition, testing at least 3 sources of retinal progenitors in vivo may uncover differences in neuroprotective efficacy, which may not be determined by proteome and transcriptome analysis. This information will be helpful for delineating the neuroprotective mechanism and finding key neuroprotective molecules (e.g., other than proteins/peptides, e.g., microRNAs).

Transcriptome, proteome and secretome (via proteomics, Mass-Spec, microRNA analysis) signature of retinal progenitors at passages 1, 2 and 3 (and potentially higher if we observe steady rise in the level of neuroprotective transcripts from P1<P2<P3 etc.) will be compared. A passage number (likely P2 but may be higher, which provides a lot of advantages for expanding and stocking the cultures) will be selected for in vivo experiments on neuroprotection in rd10 Pde6b−/− mice and Rho-mutant P23H rats. We will karyotype and DNA-fingerprint retinal progenitors at P1, P2, P3 (and higher, if P4, for example, may look more promising based on transcriptomics/proteomic profiling) to make sure the cells maintain a stable karyotype and that the graft can be traced to the original source of cells. We will also establish several large lots of passage-2 frozen retinal progenitors, prepared with the same protocol from the same organoids (same age, same origin).

We will test P2 (at different passages, as discussed above) of retinal progenitors in rd10^{Pde6b-/-} mice and Rho-mutant P23H rats by transplanting cells (several escalating dosages of 25,000 cells, 50,000 cells, 60,000 cells, 75,000 cells) in each model. rd10^{Pde6b} mice: grafted at postnatal day 30. Mice will be dark-reared PO-P30 to enable them reach young adult age without loss of PRs (e.g., using the EYECRO protocol), then grafted (intravitreally) with retinal progenitors and reared at 12 h light-12 h dark cycle. One eye (OD) will receive a graft (suspension of cells in 1.5 μl volume), while the other eye (OS) of each animal will receive conditioned medium from the same cells (same volume). The animals will be tested monthly with OCT (retinal thickness), scotopic and photopic ERG (visual/PR function), optokinetic testing (OKT) (quantification of visual acuity and contrast vision), then terminated after 3 months, the eyes fixed with 4% PFA, embedded in the optimum cutting temperature (OCT) medium and assayed for histology/IHC. Rho-mutant P23H rats: grafted (intravitreally) with retinal progenitors at postnatal day 21 (before the onset of RD) and reared at 12 h light-12 h dark cycle. One eye (OD) will receive a graft (suspension of cells in 4 μl volume), while the other eye (OS) of each animal will receive conditioned medium from the same cells (4 μl). The animals will be tested monthly with OCT (retinal thickness), scotopic and photopic ERG (visual/PR function), OKT (quantification of visual acuity and contrast vision), then terminated after 6 months, the eyes fixed with 4% PFA, embedded in the optimum cutting temperature (OCT) medium and assayed for histology/IHC.

Analysis of molecules, which are expressed by retinal progenitors in vitro and in vivo in the epiretinal (vitreous) space will help to define the preferred passage number of retinal progenitors, which may exert highest neuroprotective impact on degenerating retina.

Methods for determining the efficacy of transplantation of retinal progenitor cells isolated from stem cell derived retinal tissue may include:

Examine the vitreous of the animals for the presence of human neuroprotective molecules (proteomics, microRNA analysis, neurolipids, neuropeptides).

Dissect a portion of rodent eyes with grafts, conduct RNA-seq analysis of the rodent retina and RNA-seq analysis of the grafts to delineate the transcriptome of epiretinal grafts in vivo and the response of degenerating rodent retina to neuroprotection. This is expected to define the pathways activated/downregulated in degenerating retina, which are likely impacted by the vitreal grafts. This will detect the molecules in the secretome, which are modulating these pathways.

Engineer hESCs to carry the multicistronic (2-3 messages) vector to express our current best candidate neuroprotective factors in retinal cells, derive retinal organoids, dissociate and establish P2 culture of retinal progenitors, transplant these cells (60,000 cells/eye) in RCS or Rho-mutant P23H rats, and delineate the impact of these grafts on the progression of RD. We already have a short list of neuroprotective proteins (based on the initial analysis of transcriptome from hESC-3D retinal tissue). However, we expect that it may be more productive to test the engineered cells later in this project, when we develop better understanding about the key neuroprotective molecules in our secretome.

The safety of selected retinal progenitor preparations in a large eye animal model (rabbit) will also be demonstrated. Epiretinal grafts of retinal progenitors are expected to be most efficacious if they are placed (i) in close proximity to the degenerating retina, and if (ii) higher therapeutic dosage of cells is used (3-6×10⁶ cells in case of human eye). However, using the high cell dosage in rodent eye corresponding to this number of 3-6×10⁶ cells/eye (when adjusted for the axial length of human vs rodent eye) may not be feasible to due to large lens size in rodents, which greatly reduces vitreous chamber depth (VCD).

Rabbit models may be immunosuppressed from day-3 before grafting and throughout the whole experiment, daily, until the rabbits are terminated, with Cyclosporine A and Prednisolone (optional: add dexamethasone drops). We will graft the selected preparation of retinal progenitors (escalating dosages of 0.5×10⁶, 1×10⁶, 1.5×10⁶ cells) into the epiretinal space of young adult Dutch Belted rabbits to demonstrate that vitreal grafts do not cause any adverse impact on the recipient eye (no tumorigenesis, loss of vision, retinal detachment/ERM, inflammation etc.). We will terminate animals at 1 week, 2 months, 6 months after grafting to perform postmortem histology and IHC to delineate the presence of any signs of adverse reaction of the recipient ocular tissue to grafts (increased level of the immune cells, GFAP fibers in retina, signs of retinal cell death etc.). We will take small samples of the vitreous at different time points (from 1 week to 1 year) for proteome/secretome analysis to assay for human peptides, proteins, microRNAs, exosomes as well as inflammatory host-specific cytokines and other host-specific molecules related to inflammation. We will take the (i) whole vitreous of one rabbit (containing the human graft), and (ii) the whole 1 rabbit retina (same animal) at 1 week, 2 months, 6 months and perform (i) transcriptional/proteome profiling of the grafts and the recipient rabbit retina to further determine the level of potentially neuroprotective molecules in grafts and the response of the recipient retina to human grafts on the molecular level.

The safety protocol can include:

Test the 1^{st} cohort once by ERG, OCT, fundus exam at +1 week (wk) after grafting, and terminate.

Test the 2nd cohort once in 2 weeks (at +2 wk, +4 wk, +6 wk, +8 wk) by ERG, OCT, VEP, fundus exam, and terminate at 2 months (month) after grafting cells into the vitreous.

Test the 3rd cohort twice a month for the 1^{st} 2 month, and then once a month by ERG, OCT, VEP, fundus exam, and terminate at 6 months after grafting cells into the vitreous.

Antibodies to the following proinflammatory retinal markers for IHC: Iba-1² (microglia/activated macrophages), GFAP^{2,22}, NF-kB²¹³, CD3, CD4, CD8 may be used.

TABLE 1
Experimental Design

| Number of retinal | Terminate at +1 wk | Terminate at +2 mo | Terminate at +6 mo |
|---|---|---|---|
| progenitors, P2 | after grafting | after grafting | after grafting |
| 0.5 million | 4 rabbits (R eye cells, | 4 rabbits (R eye cells, | 4 rabbits (R eye cells, |
| 50 μl. volume | L eye control = | L eye control = | L eye control = |
| | conditioned medium) | conditioned medium) | conditioned medium) |
| 1 million | 4 rabbits (R eye cells, | 4 rabbits (R eye cells, | 4 rabbits (R eye cells, |
| 50 μl. volume | L eye control = | L eye control = | L eye control = |
| | conditioned medium) | conditioned medium) | conditioned medium) |
| 1.5 million | 4 rabbits (R eye cells, | 4 rabbits (R eye cells, | 4 rabbits (R eye cells, |
| 50 μl. volume | L eye control = | L eye control = | L eye control = |
| | conditioned medium) | conditioned medium) | conditioned medium) |

(Passage 2 retinal progenitors of the improved cell prep, engineered [at hESCs developmental stage] to express 2-3 key neuroprotective factors): 3 dosages, 3 timepoints each.

Test the 1^{st} cohort once by ERG, OCT, fundus exam at +1 wk after grafting, and terminate.

Test the 2^{nd} cohort once in 2 weeks (at +2 wk, +4 wk, +6 wk, +8 wk) by ERG, OCT, VEP, fundus exam, and terminate at 8 weeks (2 months) after grafting cells.

Test the 3^{rd} cohort twice a month for the 1^{st} 2 month, and then once a month by ERG, OCT, VEP, fundus exam, and terminate at 6 months after grafting cells.

Testing cGMP cell prep of retinal progenitors for safety: 3 dosages, 3 timepoints each.

Test the 1^{st} cohort once by ERG, OCT, fundus exam at +1 wk after grafting, and terminate.

Test the 2^{nd} cohort once in 2 weeks (at +2 wk, +4 wk, +6 wk, +8 wk) by ERG, OCT, VEP, fundus exam, and terminate at 8 weeks (2 months) after grafting cells.

Test the 3^{rd} cohort twice a month for the 1^{st} 2 month, and then once a month by ERG, OCT, VEP, fundus exam, and terminate at 6 months after grafting cells.

Interpretation of results: Changes in retinal thickness on the OCT scan may indicate retinal inflammation (retinal thickening) or/and retinal, including photoreceptor, degeneration. Loss of the inner (the ellipsoid zone) and outer segments may also be observed. The amplitude of the scotopic and photopic a-wave (rod and cone photoreceptors, respectively) and b-wave (INL) in the ERG exam in the OD (grafts) vs. OS (sham surgery) eyes of the same animal will indicate that electrophysiological function of retina is not impacted by the grafted cells. Fundus imaging is used to evaluate the overall health of retina and detect signs of retinal degeneration early, including the abnormal vasculature.

We carried out power analysis to estimate the minimum needed number of animals in each cohort for experiments. The Photopic ERG b-wave at 2 months was 14.2+/−2.79 cds/m2, while at 8 month it was 4.2+/−2.5 cds/m2. If with (cell therapy) treatment the cones are preserved (to have 100% more (i.e. twice) the number of surviving cones in the “treated” cohort), then the mean ERG would be 8.4 uV (compared to 4.2 uV for untreated). In this case the sample size should be 5 animals. If with (cell therapy) treatment the cones are preserved (to have 75% more the number of surviving cones), then the mean ERG would be 7.35 uV (compared to 4.2 uV for untreated animals). In this case the sample size should be 7. If with cell therapy treatment the cones are preserved (to have 50% more as many again in the treated eyes) then the mean ERG would be 6.3 uV (compared to 4.2 uV for untreated), which makes the sample size 13 (animals). The second option (7 animals) is a good middle ground estimate. Therefore, to make the number of males and females the same, we will use 4 males and 4 females in each cohort.

Further studies in one rodent model (most likely Rho-mutant P23H rat, as most commonly found RP mutation in patients) and one large eye model (to be determined), as well as in rabbits, using cGMP-retinal progenitors prepared from cGMP-hESC-3D retinal tissue (retinal organoids).

TABLE 2
Experimental Design

| | | Longer | | | | IND-enabling work: Pde6a |
|---|---|---|---|---|---|---|
| | Pilot | term | Crx | Aipl1 | Cngb1 | in another model - to be |
| | Pde6a | Pde6a | cat | cat | dog | determined based on results |
| Number | 8 | 8 | 8 | 8 | 8 | 8 |
| animals | | | | | | |
| OD | cells | cells | cells | cells | cells | cells |
| OS | control | control | cells (2 | control | control | control |
| | | | eyes as | | | |
| | | | control) | | | |

Control: sham-injection into vitreous, OS (left eye). Cell-free conditioned medium, equal volume. In case of Crx^{Rdv+} model, which has more uniform rate of degeneration, similar in all Crx^{Rdv+} cats (Occelli, Tran, & Petersen-Jones, 2016) we will leave only 2 control eyes and inject cells into 12 eyes.

Efficacy will be measured by improved/unchanged vision in treated eyes in small eye rodent models of RD [Rho P23H rat, rd10^{Pde6a-/-} mouse and RCS rat) and preservation of PR layer thickness. Safety of the progenitor cell graft preparations will be measured by, for example, tumorigenesis, loss of vision, retinal detachment/ERM, inflammation to demonstrate that vitreal grafts do not cause any adverse impact on the recipient eye. Safety will also be demonstrated in large-eye models starting with Pde6a−/− dog, then Aipl1^{-/-} cat, Cngb1^{-/-} dog and Crx^{+/-} cat.

### Example 7

The methods described herein for producing stem cell derived retinal tissue may be used to generate scalable production of stem cell derived retinal organoids for transplantation to a subject in need thereof. It has been shown that stem cell derived tissue generated using the improved methods described herein demonstrate inner, outer segments and cilia in photoreceptors, rods and cones with Rhodopsin, Cone Opsins and Recoverin, produces hundreds of retinal organoids, does not lead to tumorigenesis in vivo in both rats and cats for at least 6 months. It has also been shown that the methods described herein can be used with many different human embryonic cell (hECS) lines, such as but not limited to, Wisconsin H1, ESI053, ESI049, ESI017.

Stem cell derived retinal tissue and the methods for generating the same as described herein may have at least one of the following criteria:

Easy and can be done by a technician in a cGMP-facility;

Method is compatable with different cell lines and has been tested in hESC lines (Wisconsin lines H1, also Biotime's hESC lines ESI053, ESI049, ESI017; all lines have cGMP stocks);

Produces hundreds of retinal organoids when started from 10× p100 dishes of hESCs and can be further scaled up in a bioreactor;

Demonstrates retinal development, with CHX10, RX[+] and PAX6 [+] rim of developing neural retina, many BLIMP1H, NEUROD1[+] photoreceptor progenitors and [CRX]+photoreceptors;

In the 4-6-months of in vitro studies, has demonstrated photoreceptor inner-outer segment-cilia formation (by electron microscopy analysis), and dense layer of photoreceptors with Rhodopsin, Cone Opsins and Recoverin staining, and also presence of inner layer neurons (e.g., Calretinin);

cGMP-compatible (i.e., all components of the differentiation media are cGMP-compatible), and thus, can be used for making cell therapy product, when done in cGMP facility.

Improved shipping methods were also employed for shipping viable and transplantation ready stem cell derived retinal tissue.

In addition, retinal organoids derived with the methods described herein were submitted for RNA-Seq analysis. The data demonstrated high level of RAX, CHX10, other retinal markers including photoreceptors, and a level of Synaptophysin that was higher than that in organoids generated using previous methods.

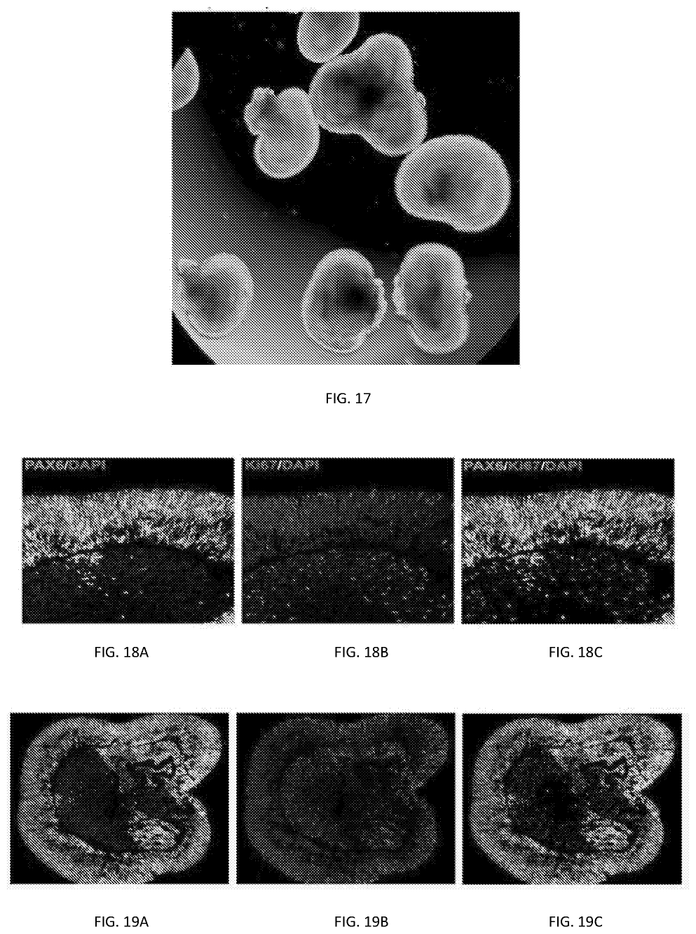

FIG. 17 shows an image of stem cell derived retinal tissue derived from the H1 (WA01) hESC line at between about 2-3 months using methods described herein. Initial derivation was carried out for several week under adherent conditions. The aggregates were then cultured under nonadherent conditions in ultra-low attachment plates.

Immunohistochemical staining of stem cell derived retinal tissue generated according to the methods described herein was used to show the distribution of cell division marker, Ki67 and PAX6 in the neural retina of stem cell derived retinal tissue at about 2.5 months after initiation of induced differentiation. As shown in FIG. 18A through FIG. 19C, Ki67 distribution in the manmade artificial retinal tissue developed herein resembles that in the developing mammalian neural retina at about 9-12 weeks of human development.

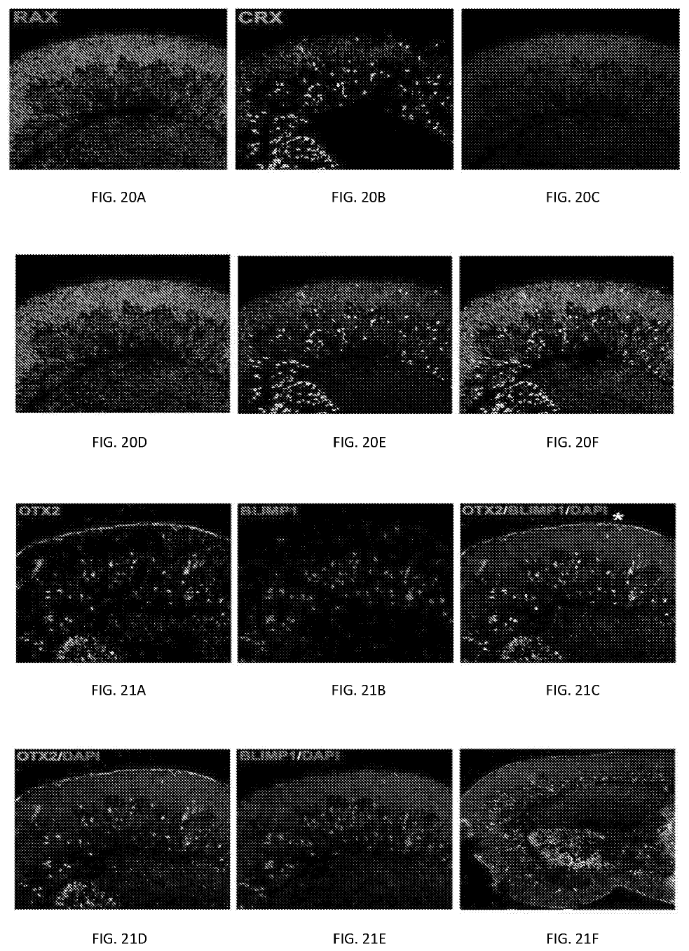

As shown in FIG. 20A through FIG. 20F, stem cell derived retinal tissue display the markers for RX (RAX, an eyefield marker), and CRX (cone-rod homeobox, photoreceptor marker), counterstained with pan-nuclear stain 4′,6-diamidino-2-phenylindole (DAPI). This is a typical staining of retinal organoids differentiated with our Protocol #3 between 2-2.5 months after initiating differentiation and shows large RAX[+]rim corresponding to developing neural retina, with many developing photoreceptors (CRX), and the RAX[+] core. A number of CRX[+] photoreceptor progenitors remain there. Organoids from ESI lines have the same distribution of RX and CRX markers (data not shown).

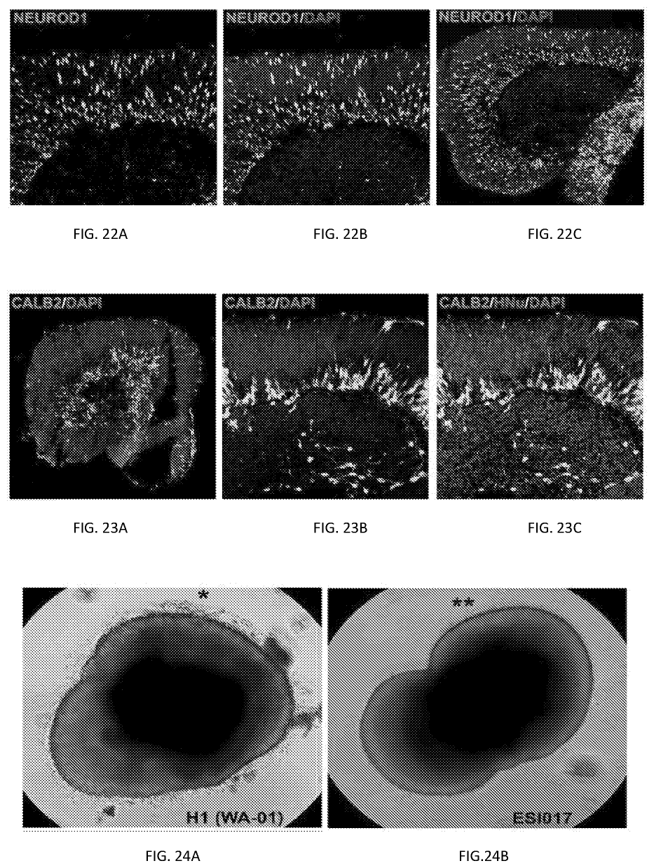

FIG. 21A through FIG. 21F are images of immunohistochemically stained hESC-3D retinal tissue (retinal organoid, frozen section) with antibodies to OTX2 (cone-rod photoreceptors and RPE) and BLIMP1, a photoreceptor progenitor marker), counterstained with pan-nuclear stain 4′,6-diamidino-2-phenylindole (DAPI). Stem cells were induced to differentiate for between 2-2.5 months and show co-localization of BLIMP1[+] and OTX2[+] photoreceptor progenitors in the rim. Photoreceptors are born in the apical side next to RPE (asterisk). A number of OTX2[+] photoreceptor progenitors remain in the central core area and fail to exit. Organoids from ESI lines have the same distribution of OTX2 and BLIMP1 markers (data not shown).

FIG. 22A through FIG. 22C are images of immunohistochemically stained hESC-3D retinal tissue (retinal organoid, frozen section), at between 2 and 2.5 months after inducing differentiation, with antibodies to NEUROD1 (photoreceptor progenitor and amacrine cell progenitor marker), counterstained with pan-nuclear stain 4′,6-diamidino-2-phenylindole (DAPI). The images show a large number of NEUROD1[+] photoreceptor/amacrine progenitors in the rim corresponding to the developing neural retina within the organoids. Organoids from ESI lines were shown to have the same distribution of RX and CRX markers (data not shown).

FIG. 23A through 23C are images of immunohistochemically stained hESC-3D retinal tissue (retinal organoid, frozen section), at between 2 and 2.5 months after inducing differentiation, with antibodies to Calretinin (Calbindin-2, amacrine cell marker), counterstained with pan-nuclear stain 4′,6-diamidino-2-phenylindole (DAPI). These images show a large number of CALB2[+] amacrine neurons in the basal side (lumen) corresponding to developing inner nuclear layer of the neural retina within the organoids. Organoids from ESI lines have the same distribution of CALB2 (data not shown).

FIG. 24A and FIG. 24B are images of about 4.5 to 5-month-old retinal organoids generated from hESC lines, H1 (WA01) and ESI017. FIG. 24A shows a retinal organoid derived from the cell line, H1 (WA-01) and FIG. 24B shows a retinal organoid derived from the cell line, ESI017.

FIG. 25A and FIG. 25B are images of about 4.5 to 5-month-old retinal organoids generated from hESC lines, H1 (WA01) and ESI017. These images show the enlarged areas marked with a single (H1) and double (ESI017) asterisks to show inner- and outer segment-like protrusions emanating from the stem cell derived retinal tissue (organoids).

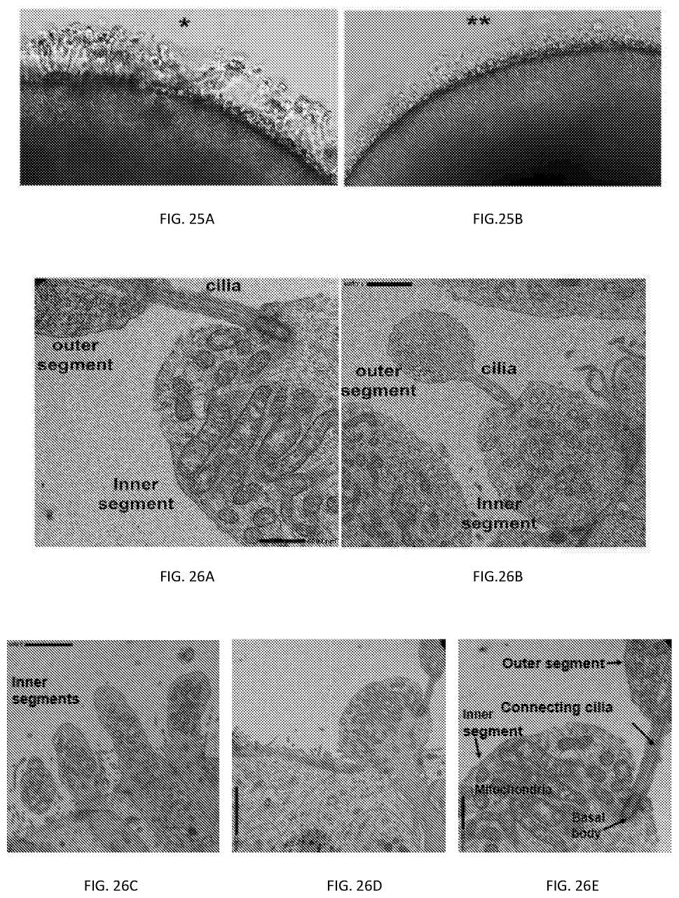

FIG. 26A through FIG. 26E are electron microscopy (EM) images of the rim of retinal organoids grown for about 5 months. Shown are inner segments, the connecting cilia and the short developing outer segments, similar to that of the developing dissociated and cultured photoreceptor cells.

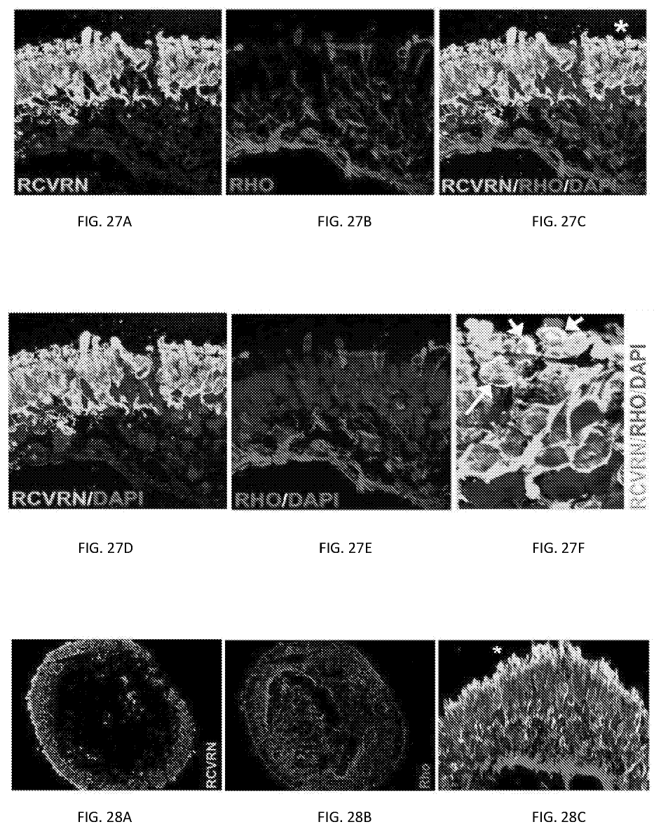

FIG. 27A through FIG. 27F are immunohistochemical images showing expression of Rhodopsin (Rho) and Recoverin (RCVRN) in retinal organoids cultured for between about 4.5 to 5 months. The confocal image of FIG. 27F demonstrates dense layer of photoreceptors in the rim of hESC-3D retinal tissue (retinal organoid) with short IS/OS-like protrusions. The asterisk in the upper right panel and the white arrows in the lower right panel (magnification of the area shown with an asterisk) point to Rhodopsin [+] cell body and IS, OS protrusions.

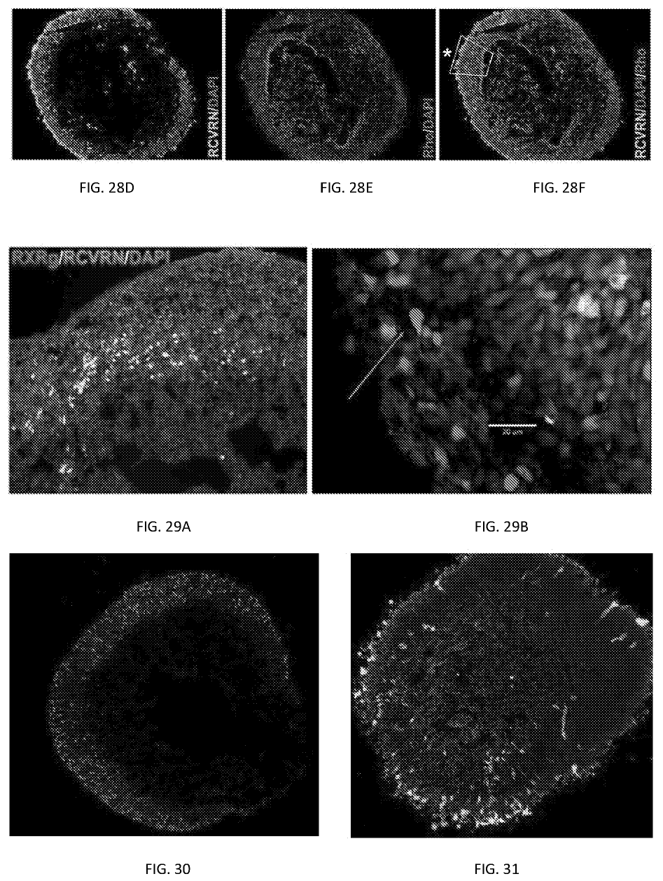

FIG. 28A through FIG. 28F are immunohistochemical images showing Rhodopsin (Rho) and Recoverin (RCVRN) staining in retinal organoids cultured for between about 4.5 and 5 months. This epifluorescent image demonstrates the distribution and the abundant presence of Rho[+] and RCVRN[+] photoreceptors in a stem cell derived retinal organoid using the methods described herein. FIG. 28C is a magnification of the retinal organoid rim, shown in FIG. 28F.

Immunohistochemistry analysis of retinal organoids was performed with anti RXRgamma and anti-Recoverin antibodies. FIG. 29A and FIG. 29B are immunohistochemical images showing young developing cone photoreceptors in the about 4-month-old retinal organoid derived from human stem cells. The data demonstrate the abundance of cone photoreceptors in stem cell derived retinal tissue (retinal organoids) according to certain embodiments described herein.

FIG. 31 is an immunohistochemical image showing developing rod photoreceptors (Rhodopsin antibody) with developing outer segments, stained with Peripherin2/RDS antibody in retinal organoid at about 4.5 months.

### Example 8

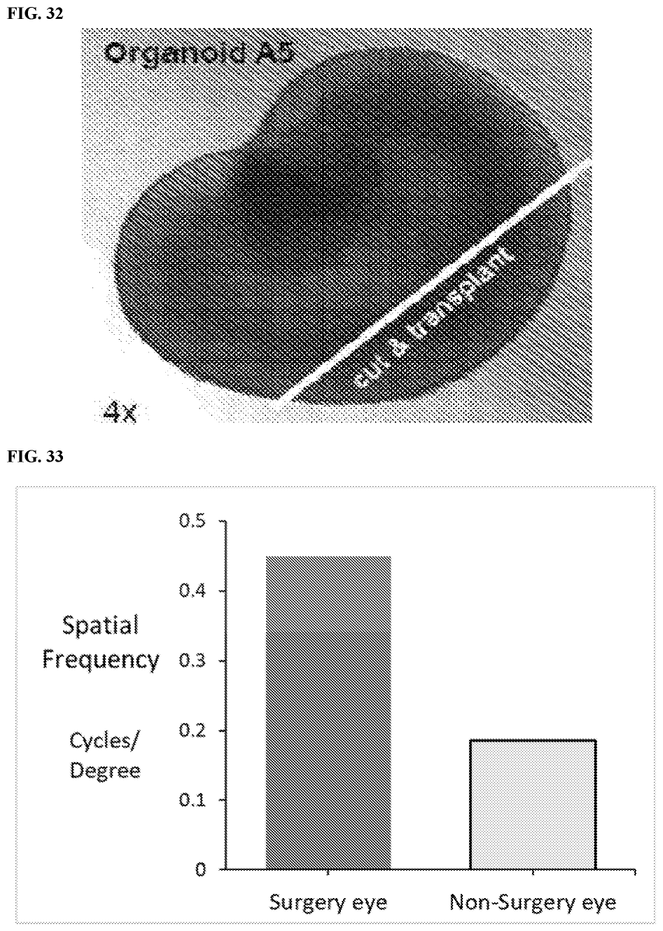

Stem cell derived retinal tissue described herein was transplanted into the subretinal space of blind T-immunodeficient SD-Foxn 1 Tg(S334ter)3Lav (RD nude) rats. Organoids were cut into sections to form pieces that comprised a portion of the rim of the organoid, which comprises may developing photoreceptors. FIG. 32 is an image of stem cell derived retinal tissue (and organoid) to be cut and transplanted into the subretinal space of a blind T-immunodeficient SD-Foxnl Tg(S334ter)3Lav (RD nude) rat.

The grafts may also have a neuroprotective impact on damaged retina (young neural tissue secreting paracrine factors). Maturation of retinal tissue and synaptic integration can take up to 6-8 months. Vision improvement was measured by correlating the optokinetic response and the response to light in the brain (the superior colliculus (SC)).

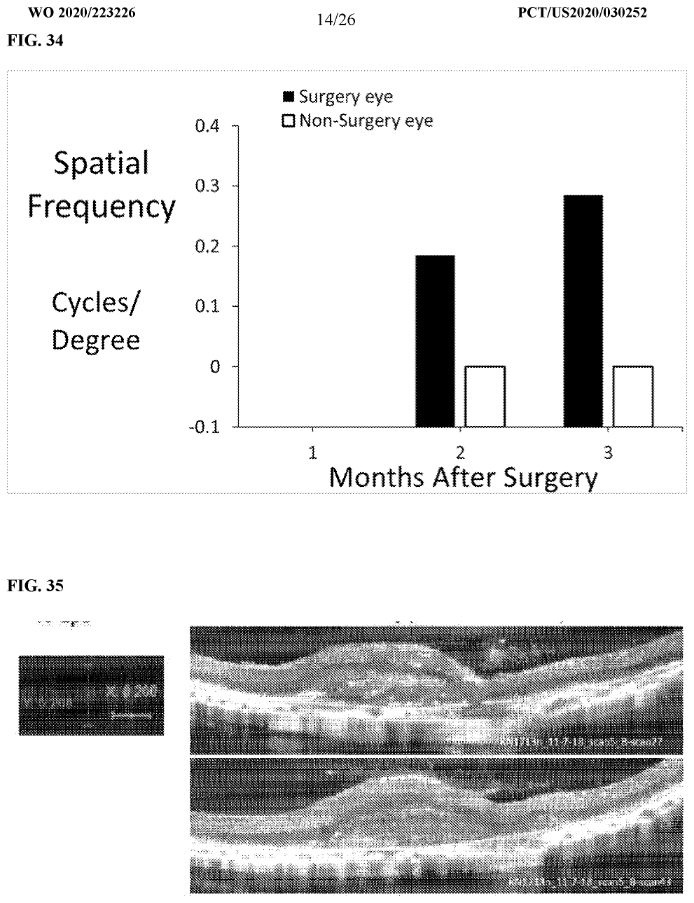

Improvements in vison in the treatment eyes after transplantation of sections of stem cell derived retinal tissue (as measured by optokinetics) was shown in both the disease model (S334ter rat) and RCS nude rat, as shown in FIG. 33 and FIG. 34.

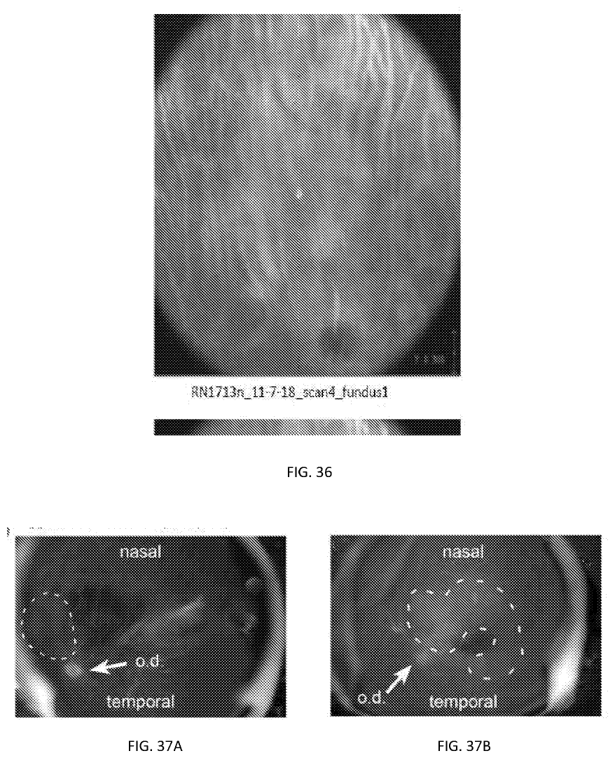

FIG. 35 is an image of an optical coherence tomography (OCT) scan of the transplanted stem cell derived retinal tissue in a rat eye. The OCT demonstrates successful grafting of hESC-3D retinal tissue into the subretinal space of immunodeficient blind rats. FIG. 36 is a fundus image that demonstrates successful grafting of hESC-3D retinal tissue into the subretinal space of immunodeficient blind rats.

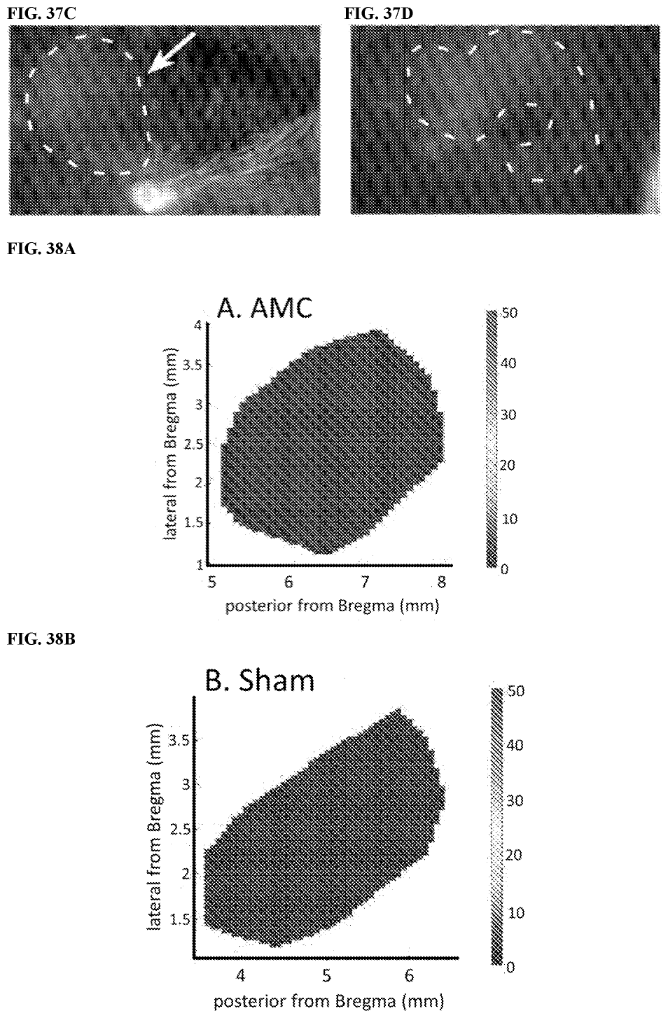

Activation of the superior colliculus (SC) in blind Rho-mutant immuno-deficient rats at about 6 months post transplantation of stem cell derived retinal tissue was demonstrated by implanting electrodes into the SC of the rats. Two controls were used: (1) an age matched control, in which no surgery prior to the activation analysis was performed and no material was implanted into the subretinal space; and (2) the Sham control, in which media was transplanted into the subretinal space instead of the stem cell derived retinal tissue. FIG. 37A through FIG. 37D show the location of the implanted retinal tissue and the implanted electrode.

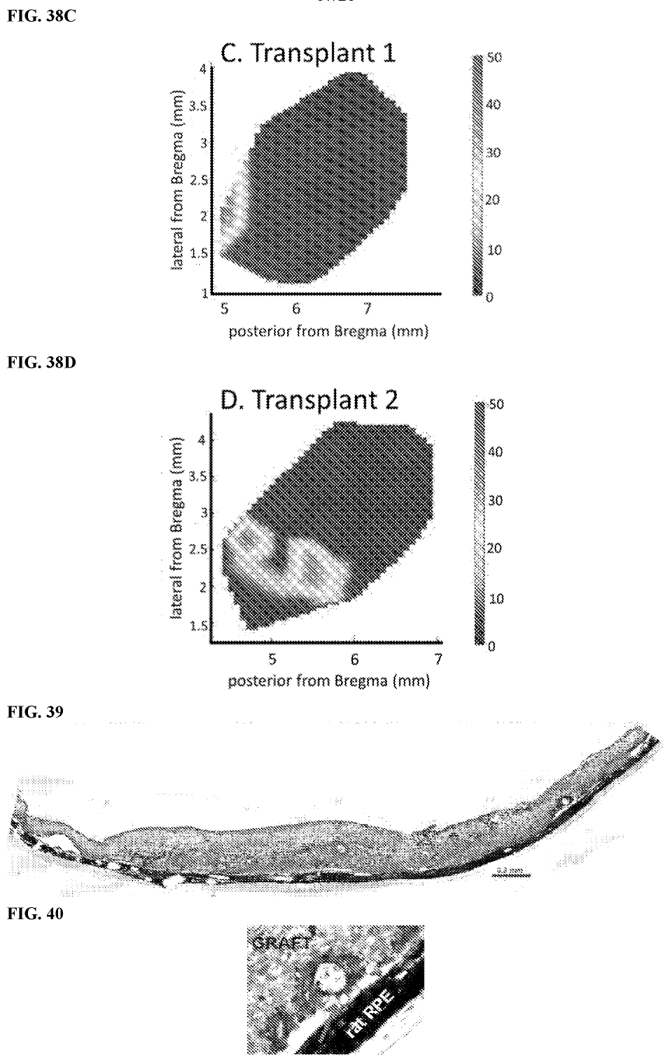

FIG. 38A through FIG. 38D show graphs of the electrical impulses generated by activation of the superior colliculus in two disease model blind rats that were treated with stem cell derived retinal tissue implants (FIG. 38C and FIG. 38D), a sham rat (FIG. 38B) and an age matched rat control (AMC) (FIG. 38A). As shown, the two treatment rats show activation of the SC. These two treatment rats demonstrated successful transplantation of the subretinal stem cell derived retinal tissue implants via OCT analysis. Sham-grafted rats showed no SC activation. Additionally, the right part of the SC (contralateral portion, which receives projection from the eye with the graft) showed activation in response to light, providing a correlation between the treatment with the stem cell derived retinal tissue and the restored vision functional outcome. Data collected at about 8 months should further demonstrate synaptic connectivity between the implanted graft and the host retina and development of rat RPE-graft photoreceptor sheet contact.

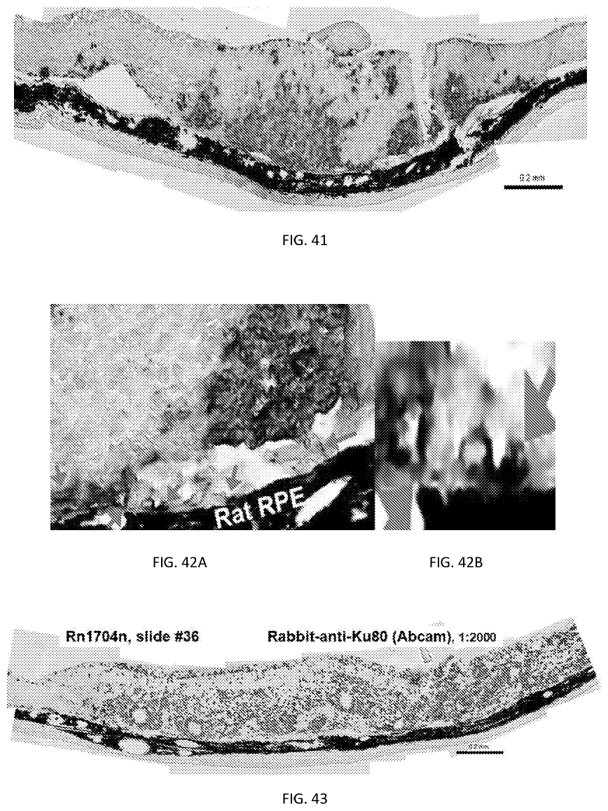

FIG. 39 is a nonfluorescent immunohistochemistry image showing the grafted human stem cell derived retinal tissue implanted into the subretinal space of a disease model rat at about 6 months. Sections were stained with rabbit anti-human recoverin. FIG. 40 is a magnified nonfluorescent immunohistochemistry image showing the grafted human stem cell derived retinal tissue implanted into the subretinal space of a disease model rat at about 6 months. Sections were stained with rabbit anti-human recoverin. Multiple rosettes of photoreceptors can be seen in the grafts with some photoreceptors forming outer segment contacts with the recipient RPE. Although rat RPE was not stained, in this disease model, the rat photoreceptors are expected to have deteriorated by this time (about 6 months). FIG. 41 is a nonfluorescent immunohistochemistry image showing the grafted human stem cell derived retinal tissue implanted into the subretinal space of another disease model rat at about 6 months. Sections were stained with rabbit anti-human recoverin. These images also demonstrate survival of the graft for at least about 6 months after implantation into a damaged or disease degenerated retina.

FIG. 42A and FIG. 42B are nonfluorescent immunohistochemistry image showing the grafted human stem cell derived retinal tissue implanted into the subretinal space of another disease model rat at about 6 months, with outer segment like protrusions from Rho positive drafts extending towards the rat RPE. Sections were stained with rabbit anti-human rhodopsin. FIG. 42B is a magnification and shows integration of the graft into the rats RPE.

FIG. 43 is a nonfluorescent immunohistochemistry image showing the grafted human stem cell derived retinal tissue implanted into the subretinal space of the same disease model rat subject (rat #1704) depicted in FIG. 40 and FIG. 41, at about 6 months. Immunohistochemical analysis of human nuclei-specific antibody Ku-80 staining indicates that the graft in the subretinal space comprises human retinal tissue, and not rat retina. Most of rat neural retina (all PRs and most INL cells except for retinal ganglion cells) are expected to have degenerated by this time. Therefore, many Rhodopsin [+] and Recoverin [+] protrusions from the grafted PRs toward rat RPE are human inner and outer segments. Synaptophysin [+] retinal tissue was also seen (data not shown). No tumors were found at at least 6 months.

Thus, the grafts are capable of establishing PR-recipient RPE contact and graft (multiple cell types)-recipient retina (RGCs and remaining INL cells) synaptic contacts. Though we do not have a continuous sheet of PRs at 6 months, the IHC data supports the electrophysiological data on superior colliculus activation. PR sheets will likely appear at between about 6-8 months post implantation due to the maturing of the graft and establishment of RPE and RGC contacts with the host, which helps to form sheets of photoreceptors in the organoid-derived grafts to restore function aspects such as but not limited to, visual perception.

### Example 9

hESC derived retinal tissue (organoids) prepared as described herein, were transplanted into blind Crx Rdy/+ cats in December 2018, and showed no tumorigenesis. Immunohistochemical analysis of about 3 months post-transplant grafts showed hundreds of human Recoverin [+], S-Opsin [+] photoreceptors, with some Rhodopsin [+] in sheets in the subretinal space of cat subjects. Initial synaptogenesis was observed with human synapses in the cat retina at about 3 months, which is earlier than expected. Further analysis will be performed at about 6-12 months in cat subretinal space.

Crx Rdy/+ cat is a model of early-onset RD (Leber Congenital Amaurosis). The loss of vision proceeds at about the same rate in all the cats of the same age, and in two eyes of the same cat. Stem cell derived retinal tissue (retinal organoids) was transplanted into both eyes of cat subjects and each eye was counted as an individual sample. Several eyes will be used as control eye samples.

The cat subjects described herein have shorter photoreceptor outer segments (OSs) due to the mutation in Crx gene and never fully develop OSs. Because of this, the neural retina and RPE have difficulty reattaching and the retinotomy/retinal bleb (needed for creating space for placing the organoids) can be very small.

The first group of Crx Rdy/+ cats (total of 2 cats, 2 months old), which received retinal organoids derived according to the methods described herein was transplanted successfully. Some cat subjects received bilateral grafts (3-4 organoids/eye), while the other cat subjects received organoid grafts in one eye (2nd eye left as control).

The third cohort of 5 cats was grafted on Mar. 2-3, 2019 and consisted of:

One 2-month old cat (bilateral grafts of H1-organoids in one eye and ESI-053 organoids in the other eye, 3-4 organoids/graft).

4× about 4-month-old cats (stem cell-derived organoids): total of 7 grafts were done, and one eye was left as control.

The fourth cohort: 6×2-mo old Crx Rdy/+ cats. 3 of the 6 cats will receive grafts (all bilateral).

Confocal immunohistochemistry was analyzed at about 3 months after stem cell derived retinal tissue was transplanted. Each cat was followed with weekly fundus exam (RetCam), while OCTs were done at 1 and about 2-2.5 months after surgery.

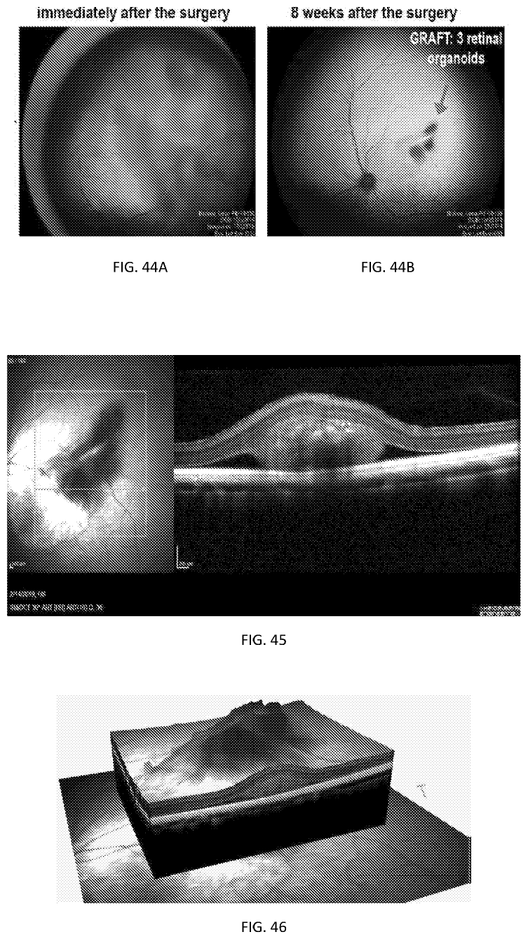

FIG. 44A and FIG. 44B are fundus images of stem cell derived retinal grafts just after implantation (FIG. 44A) and at about 2.5 months after the implantation (FIG. 44B) into the subretinal space of Crx Rdy/+ cats. Expected bleeding just after the surgery can been seen as well as a very clear RetCam image after about 2.5 months, indicating successful stem cell derived retinal tissue implantation and integration at about 2.5 months with no tumorigenesis.

FIG. 45 is an OCT image of cat eye at about 2 months and about 1 week after the implantation of the retinal tissue graft. As shown, the cat retina reattached with the RPE after implantation.

FIG. 46 is an image of a 3D reconstruction of one of the organoids in the eye shown in FIG. 45 in the cat's subretinal space, demonstrating successful grafting and reattachment of the cat retina and RPE.

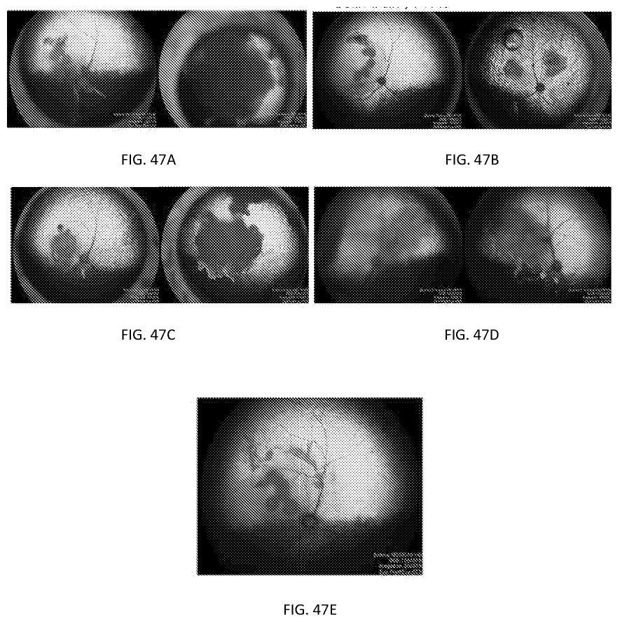

FIG. 47A through 47E are a set of RetCam images showing the successful implantation of stem cell derived retinal tissue into the subretinal space of Crx+/− cat eyes. Images were taken at about 4 months after implantation.

FIG. 48A and FIG. 48B are confocal immunohistochemical images of about 6 pieces of stem cell derived retinal organoids transplanted in to the subretinal space of a Crx Rdy/+ cat at about 3 months after implantation.

In one subject, about 6 pieces of stem cell derived retinal organoids were transplanted into the subretinal space of a Crx Rdy/+ cat. Immunosuppression was applied daily, which comprised an oral prednisolone and Cyclosporine A regimen. Confocal immunohistochemical images of the 6 stem cell derived retinal organoids transplanted into the subretinal space of a Crx Rdy/+ cat at about 3 months after implantation were taken and analyzed. Sections were stained with synaptophysin (SYP), recoverin (RCVRN), and DAPI. As shown in FIGS. 48A and 48B, human retinal organoid-derived photoreceptor clusters (RCVRN) in the subretinal space and Synaptic boutons (hSYP=Synaptophysin) in the cat INL can be seen, indicating that the PRs are maturing and there was little to no immune response. Survival can be seen for at least 3 months.

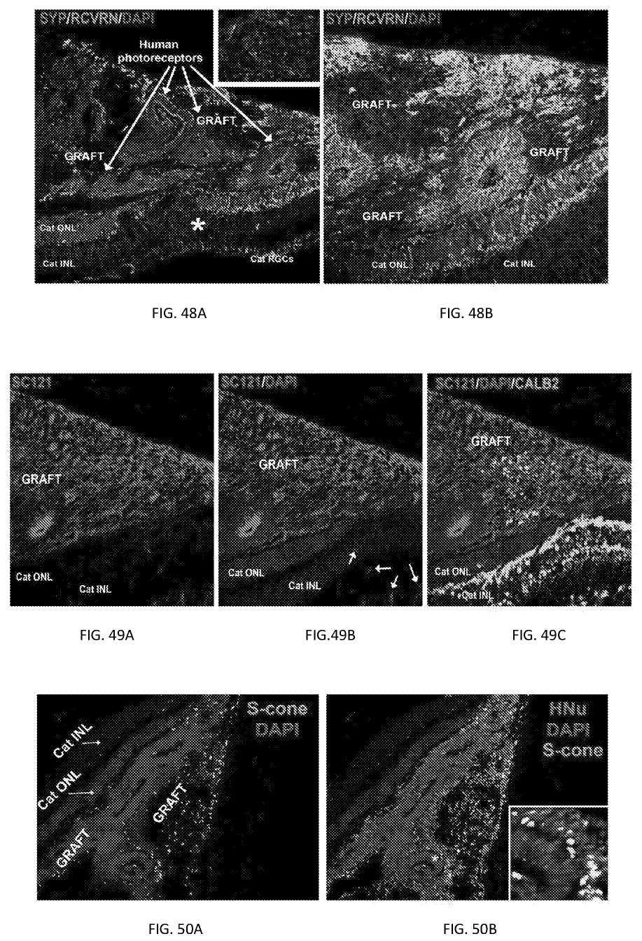

FIG. 49A through FIG. 49C are confocal immunohistochemical images showing organoid graft/cat ONL interaction. Sections are stained with SC121, calretinin and DAPI. As shown, some human SC121[+] fibers can be seen penetrating the cat ONL and cat INL. SC121 is a pan-human cytoplasm marker. Human CALB2 (Calretinin) [+] cells can be also be seen in the graft. Calretinin is found in the amacrine and horizontal cells as well as in displaced amacrine cells of the retina. The white arrows indicate signs of axonal connectivity and successful survival of graft and second order neurons.

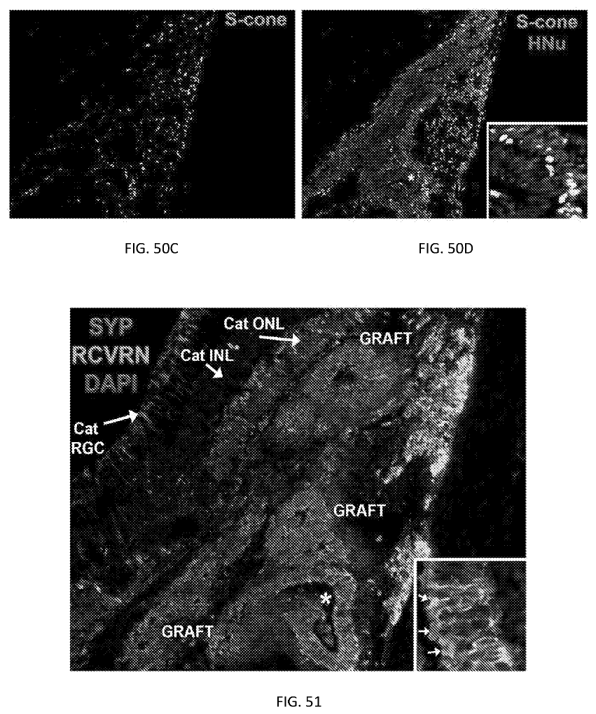

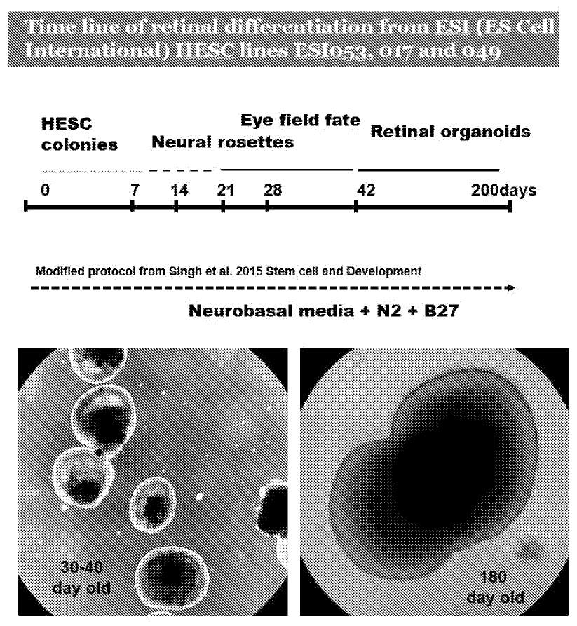

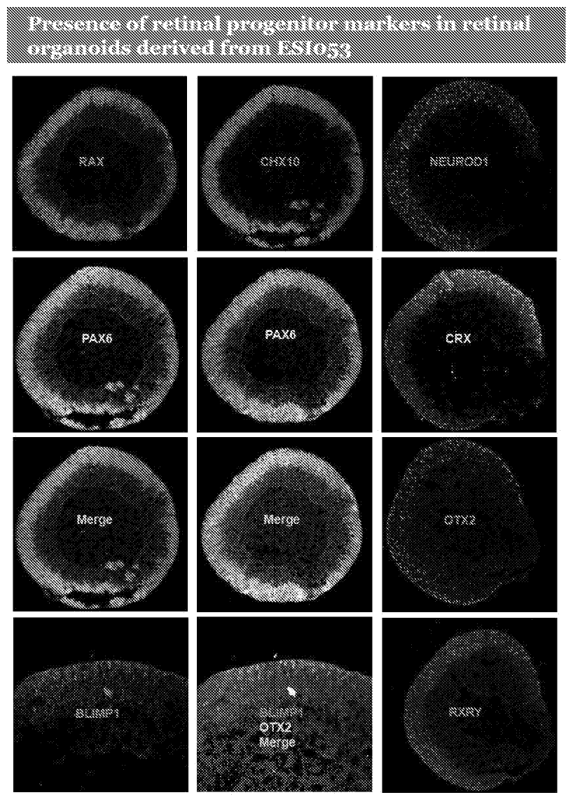

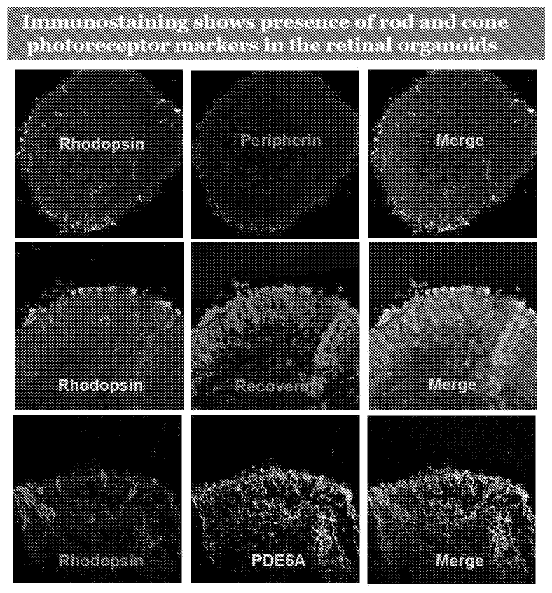

FIG. 50A through FIG. 50D are confocal immunohistochemical images showing S-cone photoreceptors in the subretinal graft. Human nuclei (HNu) antibody stains human cells but not cat cells and demonstrates the differentiation between graft tissue from host tissue. Asterisks identify the area in the main image, shown in the insets. In AMD, cone regeneration or prevention of loss can improve a subject's condition because in AMD, the macula degenerates and is comprised of mostly cones.

FIG. 51 is a confocal immunohistochemical image showing human RCVRN [+] photoreceptors in the subretinal graft, cat RCVRN [+] photoreceptors in cat ONL, and human SYP[+] (human Synaptophysin) boutons in cat INL and RGC layer. This image indicates evidence of initial synaptic connectivity between the organoid graft and host. The asterisk marks the area in the main image which is enlarged in the inset. The arrows in the inset point to short inner/outer segment protrusions in rod and cone photoreceptors, organized in sheets in the cat's subretinal space.

Tumor-free survival of sheets of transplanted human photoreceptors (cones and rods) in the cat subretinal space were demonstrated. Immunosuppression protocols worked well and prevented immune rejection of grafts by the host. Most treated eyes had several retinal organoids. Rapid closure (healing) of the retinotomy and re-connection between the cat photoreceptors and cat RPE was also shown. Sheets of human photoreceptors (cones, and also Rhodopsin [+] rods were seen (data not shown) and these photoreceptors contained small inner and outer segments. It was demonstrated that the implanted photoreceptors can form outer and inner segments after about 4-5 months in culture. Evidence of synaptic connectivity GRAFT->host was demonstrated and is expected to rapidly increase as grafts mature and spend 6 month-12 month in subretinal space. At about 6 to 12 months, it will be determined whether grafts are able to activate cat RGCs and provide visual perception to the blind cat retina.

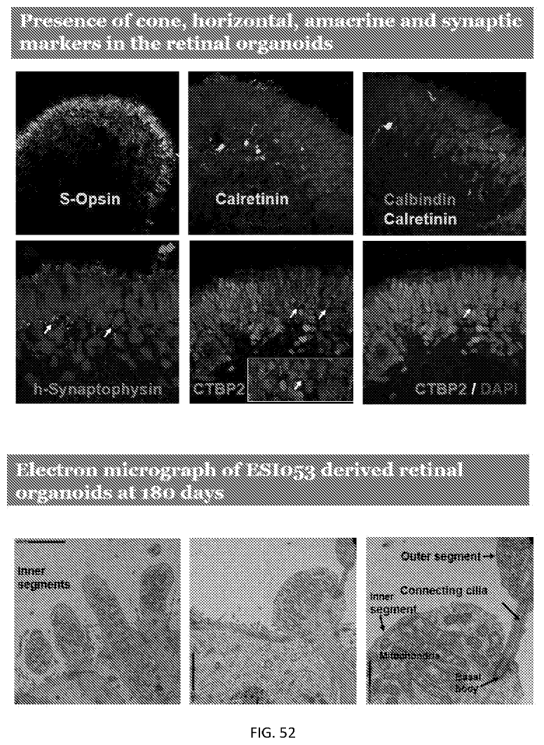

FIG. 52 is a summary of an evaluation of human embryonic stem cell lines for differentiation into three-dimensional retinal tissue (organoids) for cell therapies of retinal degenerative conditions. 3D retinal tissue was derived from five hESC cell lines using a feeder-free system and a protocol modified from Singh et al., 2015, Stem Cells & Devel. Human embryonic stem cell lines were karyotyped and fingerprinting analysis was done to assign molecular genetic identity to each line. Retinal organoids were allowed to differentiate for 8 weeks before fixing with 4% paraformaldehyde, processing for frozen immunohistochemical analysis and cutting 12 micron-thick sections. Immunohistochemistry was done to visualize the expression of retinal markers of several key retinal lineages essential for cell therapies. Cell division in hESC-3D retinal tissue was evaluated using Ki67 antibody.

Karyotype of all hESC lines were normal. Fingerprinting signature of each hESC line was developed for further identity testing for cell therapy applications. Immunohistochemical profiling of 8-week old retinal organoids derived from all hESC lines revealed strong expression of retinal progenitor markers OTX2, CRX, PAX6, BLIMP1, NEUROD1, photoreceptor markers (RCVRN, RXR Gamma), amacrine markers (CALB2, CALR) and ganglion marker (BRN3B). Retinal tissue derived from all hESC lines appeared to be similar morphologically (shown: 8-week retinal tissue, WA09 line), demonstrated initial stages of lamination (with amacrine and ganglion markers facing the basal side) and differentiated with approximately the same developmental dynamics in a dish. Long-term growth (up to several months) of retinal organoids from several lines demonstrated progressive growth and preservation of translucent color of the rim, containing developing neural retina.

These results enable testing of hESC-3D retinal tissue from ESI lines (for which cGMP-grade hESC stocks are available) in vivo in animal models with retinal degeneration for developing cell therapies to repair retina and ameliorate vision loss.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the,” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

### P-Embodiments

Embodiment P-1. A pharmaceutical composition for treating or slowing the progression of a retinal degenerative disease or disorder comprising: retinal progenitor cells isolated from stem cell derived retinal tissue; and a pharmaceutically acceptable carrier.

Embodiment P-2. The composition of Embodiment P-1, wherein the retinal progenitor cells comprise between about 0.5 million and 1.5 million cells.

Embodiment P-3. The composition of Embodiment P-1, wherein the retinal progenitor cells express one or more of the genes OPN, IL6, VEGFA, CXCL12, PTN, Lefty2, FGF9, ctgf, JAG1, NOG, KDR, Nodal, NRG1, hbergf, bmp2, ngfr, gdf11, tgfb1, MDK, cxcr4, sod1, B2M, SDF, CRABP1, SIRT2, SERPINF1, CLU, and BSG.

Embodiment P-4. A method of generating retinal progenitor cells, the method comprising: differentiating stem cells into retinal tissue in a medium comprising lectin; and dissociating the retinal tissue to isolate retinal progenitor cells.

Embodiment P-5. A method of treating or slowing the progression of a retinal disease or disorder, the method comprising, administering a therapeutically effective amount of a pharmaceutical composition comprising retinal progenitor cells isolated from stem cell derived retinal tissue.

Embodiment P-6. The method of Embodiment P-5, wherein the retinal progenitor cells express one or more of the genes OPN, IL6, VEGFA, CXCL12, PTN, Lefty2, FGF9, ctgf, JAG1, NOG, KDR, Nodal, NRG1, hbergf, bmp2, ngfr, gdf11, tgfb1, MDK, cxcr4, sod1, B2M, SDF, CRABP1, SIRT2, SERPINF1, CLU, and BSG.

What is claimed is:

1. A pharmaceutical composition for treating or slowing the progression of a retinal degenerative disease or disorder comprising:
retinal progenitor cells isolated from stem cell derived retinal tissue; and
a pharmaceutically acceptable carrier.

2. The composition of claim 1, wherein the retinal progenitor cells comprise between about 0.5 million and 1.5 million cells.

3. The composition of claim 1, wherein the retinal progenitor cells express one or more of the genes OPN, IL6, VEGFA, CXCL12, PTN, Lefty2, FGF9, ctgf, JAG1, NOG, KDR, Nodal, NRG1, hbergf, bmp2, ngfr, gdf11, tgfb1, MDK, cxcr4, sod1, B2M, SDF, CRABP1, SIRT2, SERPINF1, CLU, and BSG.

4. The composition of claim 1, wherein the composition is combined with a biomaterial.

5. A composition for treating or slowing the progression of a retinal degenerative disease or disorder comprising stem cell derived retinal tissue.

6. The composition of claim 5, wherein the stem cell derived retinal tissue comprises one or more of retinal progenitor cells and neuroprotective factors.

7. The composition of claim 5, wherein the stem cell retinal tissue is administered as a bioprosthetic patch, implant or sheet.

8. A method of generating retinal progenitor cells, the method comprising: differentiating stem cells into retinal tissue in a medium comprising lectin; and dissociating the retinal tissue to isolate retinal progenitor cells.

9. The method of claim 8, wherein differentiating stem cells comprises:
a. obtaining pluripotent stem cells;
b. culturing pluripotent stem cells in mTESR media for about 5 to about 8 days; and
c. further culturing the pluripotent stem from about day 5 or about day 8 until about day 30 in a medium comprising lectin until retinal tissue is formed.

10. The method of claim 8, wherein dissociating retinal tissue to isolate retinal progenitor cells comprises harvesting organoids by digesting with enzyme.

11. The method of claim 10, wherein the enzyme is papain.

12. The method of claim 10, wherein digesting is for about 20 min at about 37° C.

13. The method of claim 8, wherein the lectin is wheat germ agglutinin.

14. A method of treating or slowing the progression of a retinal disease or disorder, the method comprising, administering a therapeutically effective amount of a pharmaceutical composition of claim 1.

15. The method of claim 14, wherein the composition comprises stem cell derived retinal tissue.

16. The method of claim 14, wherein the composition comprises retinal progenitor cells isolated from stem cell derived retinal tissue.

17. The method of claim 14, wherein the retinal progenitor cells express one or more of the genes OPN, IL6, VEGFA, CXCL12, PTN, Lefty2, FGF9, ctgf, JAG1, NOG, KDR, Nodal, NRG1, hbergf, bmp2, ngfr, gdf11, tgfb1, MDK, cxcr4, sod1, B2M, SDF, CRABP1, SIRT2, SERPINF1, CLU, and BSG.

18. The method of claim 14, wherein the composition is administered by epiretinal or vitreal injection route.

19. The method of claim 14, wherein the retinal disease is selected from Age-related macular degeneration (AMD), geographic atrophy, retinitis pigmentosa, Leber congenital amaurosis, diabetic retinopathy, retinopathy of prematurity, ocular trauma-related retinal injuries, glaucoma, retinal degenerative disease, intermediate dry AMD, retinal detachment, retinal dysplasia, retinal atrophy, retinopathy, macular dystrophy, cone dystrophy, cone-rod dystrophy, Malattia Leventinese, Doyne honeycomb dystrophy, Sorsby's dystrophy, pattern/butterfly dystrophies, Best vitelliform dystrophy, North Carolina dystrophy, central areolar choroidal dystrophy, angioid streaks, toxic maculopathy, Stargardt disease, pathologic myopia, and macular degeneration.
